(19) United States
(12) Patent Application Publication
(10) Pub. No.: US 2014/0371165 A1
(43) Pub. Date: Dec. 18, 2014

(54) Identification of NSC23925 Isomers to Reverse Multidrug Resistance in Human Cancers

(71) Applicant: THE GENERAL HOSPITAL CORPORATION, Boston, MA (US)

(72) Inventors: Zhenfeng Duan, Cambridge, MA (US); Francis J. Hornicek, Burlington, MA (US)

(73) Assignee: THE GENERAL HOSPITAL CORPORATION, Boston, MA (US)

(21) Appl. No.: 14/375,656

(22) PCT Filed: Feb. 22, 2013
(86) PCT No.: PCT/US13/27276

§ 371 Date: Jul. 30, 2014

Related U.S. Application Data
(60) Provisional application No. 61/601,782, filed on Feb. 22, 2012.

(51) Int. Cl. C07D 401/06; C07B 53/00; A61K 31/337; A61K 31/4709; A61K 31/704

(52) U.S. Cl. CPC C07D 401/06; A61K 31/4709; A61K 31/704; A61K 31/337; C07B 53/00
USPC 514/34; 546/173; 514/314

(57) ABSTRACT

This disclosure features optically active stereoisomers of (2-(4-methoxy)quinolin-4-yl)(piperidin-2-yl)methanol that reduce drug resistance, compositions containing the same, and methods of using and preparing the same.

### CROSS REFERENCE TO RELATED APPLICATIONS

This application claims the benefit of U.S. Provisional Application No. 61/601,782, filed on Feb. 22, 2012, which is incorporated herein by reference in its entirety.

### TECHNICAL FIELD

This disclosure features optically active stereoisomers of (2-(4-methoxy)quinolin-4-yl)(piperidin-2-yl)methanol (or pharmaceutically acceptable salts thereof), which are useful for reducing drug resistance, compositions containing the compounds or salts, and methods of using and preparing the compounds or salts.

### BACKGROUND

The treatment of cancer with chemotherapeutic drugs is frequently impaired or ineffective as a result of drug resistance of tumor cells. In these cases, tumors can be resistance to a variety of anticancer drugs with different structures and mechanisms of action. This phenomenon is termed multidrug resistance (MDR). Although there are several different mechanisms associated with the development of MDR, a common cause is believed to be overexpression of a plasma membrane glycoprotein (Pgp), an MDR1 gene product. This MDR1 gene product belongs to the ABC (ATP binding cassette) superfamily of transporter proteins, and it acts as an energy-dependent drug efflux pump, preventing adequate intracellular accumulation of a broad range of cytotoxic drugs including anthracyclines (doxorubicin, daunorubicin), vinca alkaloids (vincristine, vinblastine), taxanes (paclitaxel, docetaxel) and many others for cell kill. Therefore, this underlines the critical importance of identifying compounds that could inhibit Pgp activities.

WO 2010/083385 discloses methods for reducing drug resistance in a subject undergoing cancer treatment, which include administering to a subject in need thereof an effective amount of (2-(4-methoxy)quinolin-4-yl)(piperidin-2-yl)methanol (also as “NSC23295”) or a pharmaceutically acceptable salt thereof, thereby reducing drug resistance in the subject. “NSC 23925” is available from the Structural Diversity Set, which is a library of approximately 2,000 small molecules derived from the almost 140,000 compounds available on plates through the National Cancer Institute. The identifier “NSC 23925” is a depositor-supplied synonym for (2-(4-methoxyphenyl)-4-quinolinyl)(2-piperidinyl)methanol), which has the following chemical structure:

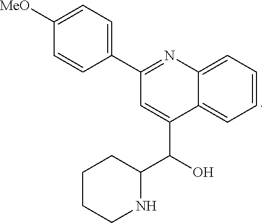 [SMILES: COc1ccc(-c2cc(C(O)C3CCCCN3)c3ccccc3n2)cc1]

As can be seen, (2-(4-methoxy)quinolin-4-yl)(piperidin-2-yl)methanol includes two stereogenic centers as part of its chemical structure. As such, there are 2² or 4 possible stereoisomers for (2-(4-methoxy)quinolin-4-yl)(piperidin-2-yl)methanol, namely two pairs of enantiomers, sometimes collectively referred to collectively as the erythro pair and threo pair; or sometimes referred to collectively as the R,R/S,S pair and the R,S/S,R pair.

### SUMMARY

This disclosure features optically active stereoisomers of (2-(4-methoxy)quinolin-4-yl)(piperidin-2-yl)methanol (or pharmaceutically acceptable salts thereof) that are useful for reducing drug resistance (e.g., reducing multidrug resistance (MDR)), in a subject (e.g., a subject having cancer, a subject having cancer and undergoing cancer chemotherapy); compositions containing the compounds or salts, and methods of using and preparing the compounds or salts.

More particularly, this disclosure provides methods for the synthesis, separation, and isolation of each of the four possible stereoisomers of (2-(4-methoxy)quinolin-4-yl)(piperidin-2-yl)methanol. This disclosure also provides methods for analyzing the chemical purity and the stereochemical purity of a sample that includes any one, two, three, or four of the four possible stereoisomers of (2-(4-methoxy)quinolin-4-yl)(piperidin-2-yl)methanol (e.g., a sample of NSC 23925 from the Structural Diversity Set library). This disclosure also features compositions (e.g., pharmaceutical compositions, medicaments) that include any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11) and one or more pharmaceutically acceptable carriers. This disclosure further provides methods for reducing multidrug resistance (MDR), e.g., reversing Pgp-mediated MDR, in a subject diagnosed with cancer and, in some implementations of the methods described herein, is also undergoing cancer chemotherapy. The methods include administering to the subject a therapeutically effective amount of any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or a pharmaceutically acceptable salt thereof.

As used herein, and for ease of exposition only, the use of the systematic name “(2-(4-methoxy)quinolin-4-yl)(piperidin-2-yl)methanol” without incorporation of stereochemical descriptors (e.g., R or S); and the use of “NSC 23925” without reference to the Structural Diversity Set are both intended to refer collectively to the group (in any ratio) of four possible stereoisomers of (2-(4-methoxy)quinolin-4-yl)(piperidin-2-yl)methanol. Each of the individual stereoisomers of (2-(4-methoxy)quinolin-4-yl)(piperidin-2-yl)methanol will be referred to herein as “NSC 23925” followed by the suffix “a”, “b”, “c”, or “d” or using a stereochemical identifier prefix, e.g., S,R (as defined herein). NSC 23925b is sometimes referred to as “11” or “compound 11.”

In some implementations of the compositions and methods described herein, a particular stereoisomer of (2-(4-methoxy)quinolin-4-yl)(piperidin-2-yl)methanol can be in purified form. Such implementations can include one or more of the following.

In some implementations of the compositions and methods described herein, a particular stereoisomer of (2-(4-methoxy)quinolin-4-yl)(piperidin-2-yl)methanol can be free (e.g., substantially free) of any one or more of the other three possible stereoisomers of (2-(4-methoxy)quinolin-4-yl)(piperidin-2-yl)methanol. Accordingly, in certain implementations, the compositions and methods described herein can include fewer than the four possible (2-(4-methoxy)quinolin-4-yl)(piperidin-2-yl)methanol stereoisomers (e.g., the compositions and methods described herein can include one or two, e.g., one, of the four possible stereoisomers).

As used herein, the designations “R,R;” “S,S;” “R,S;” and “S,R” refer to the C-17 (tertiary sp³ carbon attached to the hydroxyl group) stereochemical configuration and the C-18 (tertiary sp³ carbon attached to the ring nitrogen atom) stereochemical configuration, respectively.

In some implementations, a particular stereoisomer of (2-(4-methoxy)quinolin-4-yl)(piperidin-2-yl)methanol (e.g., the S,R stereoisomer) is free (e.g., substantially free) of its enantiomer (i.e., the R,S stereoisomer in this example).

In other implementations, a particular stereoisomer of (2-(4-methoxy)quinolin-4-yl)(piperidin-2-yl)methanol (e.g., the S,R stereoisomer) is free (e.g., substantially free) of one or both of its corresponding diastereomers (i.e., the R,R and/or the S,S stereoisomer in this example).

In still other implementations, a particular stereoisomer of (2-(4-methoxy)quinolin-4-yl)(piperidin-2-yl)methanol (e.g., the S,R stereoisomer) is free (e.g., substantially free) of its enantiomer (i.e., the R,S stereoisomer in this example) and is free (e.g., substantially free) of one or both of its corresponding diastereomers (i.e., the R,R and/or the S,S stereoisomer in this example).

In some implementations, a particular stereoisomer of (2-(4-methoxy)quinolin-4-yl)(piperidin-2-yl)methanol (e.g., the S,S stereoisomer) is free (e.g., substantially free) of its enantiomer (i.e., the R,R stereoisomer in this example).

In other implementations, a particular stereoisomer of (2-(4-methoxy)quinolin-4-yl)(piperidin-2-yl)methanol (e.g., the S,S stereoisomer) is free (e.g., substantially free) of one or both of its corresponding diastereomers (i.e., the S,R and/or the R,S stereoisomer in this example).

In still other implementations, a particular stereoisomer of (2-(4-methoxy)quinolin-4-yl)(piperidin-2-yl)methanol (e.g., the S,S stereoisomer) is free (e.g., substantially free) of its enantiomer (i.e., the R,R stereoisomer in this example) and is free (e.g., substantially free) of one or both of its corresponding diastereomers (i.e., the S,R and/or the R,S stereoisomer in this example).

As used herein, when a particular stereoisomer of (2-(4-methoxy)quinolin-4-yl)(piperidin-2-yl)methanol is described as being “free of its enantiomer” or “free of one or both of its corresponding diastereomers,” this means that the ratio of the first mentioned stereoisomer of (2-(4-methoxy)quinolin-4-yl)(piperidin-2-yl)methanol to the second mentioned stereoisomer(s) (e.g., its enantiomer) is greater than unity.

As used herein, when a particular stereoisomer of (2-(4-methoxy)quinolin-4-yl)(piperidin-2-yl)methanol is described as being “substantially free of its enantiomer” or “substantially free of one or both of its corresponding diastereomers,” this means that the ratio of the first mentioned stereoisomer of (2-(4-methoxy)quinolin-4-yl)(piperidin-2-yl)methanol to the second mentioned stereoisomer(s) (e.g., its enantiomer) is of a value that is greater than or equal to 95:5 (e.g., including but not limited to greater than or equal to 96:4; greater than or equal to 97:3; greater than or equal to 98:2; greater than or equal to 99:1; or greater than or equal to 99.5:0.5).

In some implementations, a particular stereoisomer of (2-(4-methoxy)quinolin-4-yl)(piperidin-2-yl)methanol is free or substantially free of other compound(s) (non-stereoisomers), substances, or impurities. The definitions of free or substantially free used above also apply here with respect to the ratio of the particular stereoisomer and the other compound(s), substances, or impurities.

In some implementations of the compositions and methods described herein, the number and amounts of the (2-(4-methoxy)quinolin-4-yl)(piperidin-2-yl)methanol stereoisomers are other than the number and amounts of the (2-(4-methoxy)quinolin-4-yl)(piperidin-2-yl)methanol stereoisomers (and optionally other substances) that are present in NSC 23925 from the Structural Diversity Set library. Such a determination can be made using, e.g., the methods described herein (see Examples section and HPLC traces included in the accompanying drawings. As shown in FIG. 8 (and in conjunction with FIGS. 6A-6F and FIGS. 9-13), NSC 23925 from the Structural Diversity Set library includes some amount of each of the four possible (2-(4-methoxy)quinolin-4-yl)(piperidin-2-yl)methanol stereoisomers.

In one aspect, a compound having the following formula, or a pharmaceutically acceptable salt thereof, is featured:

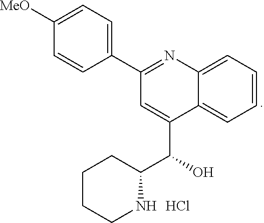 [SMILES: COc1ccc(-c2cc([C@H](O)[C@H]3CCCCN3)c3ccccc3n2)cc1 ; Cl]

This compound is sometimes referred herein as NSC 23925b or compound 11).

Implementations can include one or more of the following features.

NSC 23925b is free (e.g., substantially free) of its enantiomer

NSC 23925b is free (e.g., substantially free) of one or both of its corresponding diastereomers.

NSC 23925b is free (e.g., substantially free) of its enantiomer and is free (e.g., substantially free) of one or both of its corresponding diastereomers.

The compound has an optical rotation of about [α]_{D}²⁰=+15.0 at a concentration of 0.2 grams/100 mL of methanol

The compound is in crystalline form. In certain implementations, the crystalline compound exhibits an x-ray crystal structure substantially the same as that shown in Appendix B, which is expressly considered as part of this disclosure.

In another aspect, a compound having the following formula, or a pharmaceutically acceptable salt thereof, is featured:

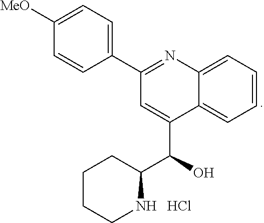 [SMILES: COc1ccc(-c2cc([C@@H](O)[C@@H]3CCCCN3)c3ccccc3n2)cc1 ; Cl]

This compound is sometimes referred herein as NSC 23925a.

Implementations can include one or more of the following features.

NSC 23925a is free (e.g., substantially free) of its enantiomer

NSC 23925a is free (e.g., substantially free) of one or both of its corresponding diastereomers.

NSC 23925a is free (e.g., substantially free) of its enantiomer and is free (e.g., substantially free) of one or both of its corresponding diastereomers.

In further aspect, a compound having the following formula, or a pharmaceutically acceptable salt thereof, is featured:

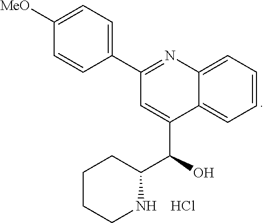 [SMILES: COc1ccc(-c2cc([C@@H](O)[C@H]3CCCCN3)c3ccccc3n2)cc1 ; Cl]

This compound is sometimes referred herein as NSC 23925c.

Implementations can include one or more of the following features.

NSC 23925c is free (e.g., substantially free) of its enantiomer

NSC 23925c is free (e.g., substantially free) of one or both of its corresponding diastereomers.

NSC 23925c is free (e.g., substantially free) of its enantiomer and is free (e.g., substantially free) of one or both of its corresponding diastereomers.

The compound has an optical rotation of about [α]_{D}²⁰=−15.8 at a concentration of 0.2 grams/100 mL of methanol

In further aspect, a compound having the following formula, or a pharmaceutically acceptable salt thereof, is featured:

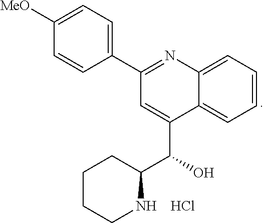 [SMILES: COc1ccc(-c2cc([C@H](O)[C@@H]3CCCCN3)c3ccccc3n2)cc1 ; Cl]

This compound is sometimes referred herein as NSC 23925d.

Implementations can include one or more of the following features.

NSC 23925d is free (e.g., substantially free) of its enantiomer

NSC 23925d is free (e.g., substantially free) of one or both of its corresponding diastereomers.

NSC 23925d is free (e.g., substantially free) of its enantiomer and is free (e.g., substantially free) of one or both of its corresponding diastereomers.

The compound has an optical rotation of about [α]_{D}²⁰=+15.6 at a concentration of 0.2 grams/100 mL of methanol

In one aspect, compositions are featured, which include any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or a pharmaceutically acceptable salt thereof, and at least one pharmaceutically acceptable carrier. Implementations can include any one or more of the featured described above and/or hereinafter.

In one aspect, methods of reducing drug resistance in a subject are featured, which include administering to the subject a therapeutically effective amount of any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or a pharmaceutically acceptable salt thereof. Implementations can include any one or more of the featured described above and/or hereinafter.

In one aspect, any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salts thereof, are featured for use in therapy. Implementations can include any one or more of the featured described above and/or hereinafter.

In one aspect, any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salts thereof, are featured for use in the preparation of a medicament for use in therapy. Implementations can include any one or more of the featured described above and/or hereinafter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

The details of one or more implementations are set forth in the accompanying drawings and the description below. Other features and advantages will be apparent from the description and drawings, and from the claims.

### DESCRIPTION OF DRAWINGS

FIGS. 1A-D depict line graphs of the effect of NSC23925 diastereomers on reverse drug resistance in SKOV-3TR cell line. Cells were treated with the paclitaxel and (2-(4-Methoxyphenyl)quinolin-4-yl)(piperidin-2-yl)methanol (also known as “NSC23925”) diastereomers NSC23925a (FIG. 1A), NSC23925b (FIG. 1B), NSC23925c (FIG. 1C) and NSC23925d (FIG. 1D) in RPMI1640 complete media at the indicated concentrations. The relative sensitivity of each line to paclitaxel was determined by MTT analysis 6 days post-treatment.

FIGS. 2A-F depict line graphs of reverse multidrug resistance by NSC23925b in MCF-7adr and SW480TR cell lines. MCF-7adr and SW480TR cells were treated with paclitaxel (FIGS. 2A-B), doxorubicin (FIGS. 2C-D) or mitoxantrane (FIGS. 2E-F) and NSC23925b in RPMI1640 complete media at the indicated concentrations. The relative sensitivity of each line to the indicated drugs was determined by MTT analysis 6 days post-treatment.

FIG. 3 depicts fluorescence images of the calcein AM efflux from NSC23925b treated MCF-7adr cell line cells.

FIG. 4A depicts ELISA assay images of cell death induced by NSC23925b in combination with doxorubicin on MCF-7adr and SW480_{TR} cell.

FIG. 4B depicts ELISA assay images of cell apoptosis induced by NSC23925b in combination with doxorubicin on MCF-7adr and SW480_{TR} cells.

FIGS. 5A-B depict images showing the effect of combination treatment with NSC23925b and paclitaxel on tumor growth in vivo.

FIG. 6A is a chiral HPLC trace of a purified sample of synthetically prepared NSC 23925a.

FIG. 6B is a chiral HPLC trace of a purified sample of synthetically prepared NSC 23925b.

FIG. 6C is a chiral HPLC trace of a mixture containing purified, synthetically prepared NSC 23925a and purified, synthetically prepared NSC 23925b.

FIG. 6D is a chiral HPLC trace of a purified sample of synthetically prepared NSC 23925c.

FIG. 6E is a chiral HPLC trace of a purified sample of synthetically prepared NSC 23925d.

FIG. 6F is a chiral HPLC trace of a mixture containing purified, synthetically prepared NSC 23925c and purified, synthetically prepared NSC 23925d.

FIG. 7 is an LCMS trace showing the chemical purity of NSC 23925 from the Structural Diversity Set library.

FIG. 8 is a chiral HPLC trace of NSC 23925 from the Structural Diversity Set library.

FIG. 9 is a chiral HPLC trace of a purified sample of synthetically prepared NSC 23925a. This trace was obtained using the same set of conditions used to obtain the trace shown in FIG. 8.

FIG. 10 is a chiral HPLC trace of a purified sample of synthetically prepared NSC 23925b. This trace was obtained using the same set of conditions used to obtain the trace shown in FIG. 8.

FIG. 11 is a chiral HPLC trace of a purified sample of synthetically prepared NSC 23925c. This trace was obtained using the same set of conditions used to obtain the trace shown in FIG. 8.

FIG. 12 is a chiral HPLC trace of a purified sample of synthetically prepared NSC 23925d. This trace was obtained using the same set of conditions used to obtain the trace shown in FIG. 8.

FIG. 13 is a chiral HPLC trace of a mixture containing purified, synthetically prepared NSC 23925a, purified, synthetically prepared NSC 23925b, purified, synthetically prepared NSC 23925c, and purified, synthetically prepared NSC 23925d. This trace was obtained using the same set of conditions used to obtain the trace shown in FIG. 8.

FIG. 14 shows the chemical structure of NSC 23925b with absolute stereochemistry as determined by x-ray crystallography. The image includes perspective views showing 50% probability displacement ellipsoids. As can be seen, C-17 in NSC 23925b has the S-configuration, and C-18 in NSC 23925b has the R-configuration.

FIG. 15 shows the three-dimensional supramolecular architecture of NSC 23925b viewed along the a-axis direction (as determined by x-ray crystallography).

FIGS. 16 and 17 are flow diagrams that summarize methods used to obtain NSC 23925b in crystalline form.

FIG. 18 is a graph showing the time course of the development of drug resistance in OVCAR8 (ovarian carcinoma) cell lines elected with taxol alone (round dots) or taxol with 1 mM compound 11 (NSC 23925b) (squares).

FIG. 19 is a graph showing IC₅₀ of taxol in parental, OVCAR8TR, and different selected cells with and without compound 11 (NSC 23925b).

FIG. 20 is a graph showing the effects of compound 11 (NSC 23925b) on reverse drug resistance in different selected OVCAR8 cell sublines.

FIG. 21 shows, in part, related proteins level in selected cell sublines.

### DETAILED DESCRIPTION

This disclosure features optically active stereoisomers of (2-(4-methoxy)quinolin-4-yl)(piperidin-2-yl)methanol (or pharmaceutically acceptable salts thereof) that are useful for reducing drug resistance (e.g., reducing multidrug resistance (MDR)), in a subject (e.g., a subject having cancer, a subject having cancer and undergoing cancer chemotherapy); compositions containing the compounds or salts, and methods of using and preparing the compounds or salts.

More particularly, this disclosure provides methods for the synthesis, separation, and isolation of each of the four possible stereoisomers of (2-(4-methoxy)quinolin-4-yl)(piperidin-2-yl)methanol. This disclosure also provides methods for analyzing the chemical purity and the stereochemical purity of a sample that includes any one, two, three, or four of the four possible stereoisomers of (2-(4-methoxy)quinolin-4-yl)(piperidin-2-yl)methanol (e.g., a sample of NSC 23925 from the Structural Diversity Set library). This disclosure also features compositions (e.g., pharmaceutical compositions, medicaments) that include any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11) and one or more pharmaceutically acceptable carriers. This disclosure further provides methods for reducing multidrug resistance (MDR), e.g., reversing Pgp-mediated MDR, in a subject diagnosed with cancer and, in some implementations of the methods described herein, is also undergoing cancer chemotherapy. The methods include administering to the subject a therapeutically effective amount of any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or a pharmaceutically acceptable salt thereof.

In some implementations, the compound has an optical purity of at least 90% diastereomeric excess.

In some implementations, the compound has an optical purity of at least 95% diastereomeric excess.

In some implementations, the compositions described herein further include another therapeutic agent, e.g., an anti-cancer therapeutic agent.

In some implementations, the anti-cancer therapeutic agent is selected from the group consisting of asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, and zalypsis.

In some implementations, the anti-cancer therapeutic agent is selected from the group consisting of paclitaxel, doxorubicin, docetaxel, calcein-AM, daunorubicin, gemcitabine, rhodamine-123, ET-743, vincristin and zalypsis.

In some implementations, the methods further include administering to the subject an anticancer therapeutic agent.

In some implementations, the subject is a patient diagnosed with cancer and undergoing cancer treatment.

In some implementations, the one or more (e.g., one or two, e.g., one) possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salt thereof, and the anti-cancer therapeutic agent are administered simultaneously.

In some implementations, the one or more (e.g., one or two, e.g., one) possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salt thereof, is administered to the subject prior to the administration of the anti-cancer therapeutic agent or is administered to the subject after the administration of said anti-cancer therapeutic agent.

In some implementations, the anti-cancer therapeutic agent is selected from the group consisting of asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, and zalypsis.

In some implementations, The anti-cancer therapeutic agent is selected from the group consisting of paclitaxel, doxorubicin, docetaxel, calcein-AM, daunorubicin, gemcitabine, rhodamine-I 23, ET-743, vincristin and zalypsis.

In some implementations, the anti-cancer therapeutic agent is a cytotoxic drug selected from the group consisting of anthracyclines, vinca alkaloids and taxanes.

In some implementations, the cancer treatment is for a cancer of breast, respiratory tract, brain, reproductive organs, digestive tract, urinary tract, eye, liver, skin, head and neck, thyroid, parathyroid or a distant metastasis of a solid tumor.

In some implementations, the distant metastasis of a solid tumor is sarcoma.

In another aspect methods of treating cancer in a subject are featured, which include: identifying a subject having developed or susceptible to developing drug resistance; administering to said subject an effective amount of any one, two, three, or four (e.g., one or two, e.g., one) of the four possible stereoisomers of (2-(4-methoxy)quinolin-4-yl)(piperidin-2-yl)methanol (e.g., S,R stereoisomer, sometimes referred to as NSC 23925b or compound 11), or a pharmaceutically acceptable salt thereof, wherein said effective amount is sufficient to reduce drug resistance in the subject, thereby treating cancer in the subject.

In some implementations, the subject is a patient undergoing cancer treatment.

In some implementations, the method further comprises discontinuing said cancer treatment.

In some implementations, the cancer treatment is discontinued prior to the administration of the one or more (e.g., one or two, e.g., one) possible stereoisomers of (2-(4-methoxy)quinolin-4-yl)(piperidin-2-yl)methanol, or pharmaceutically acceptable salt thereof.

In some implementations, the cancer treatment is discontinued after the administration of the one or more (e.g., one or two, e.g., one) possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salt thereof.

In some implementations, the method further comprises administering to said subject a subsequent cancer treatment after the discontinuation of said cancer treatment and the administration of the one or more (e.g., one or two, e.g., one) possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salt thereof.

In some implementations, the subsequent cancer treatment is the same as said discontinued cancer treatment.

In some implementations, the subsequent cancer treatment is different from said discontinued cancer treatment.

In some implementations, the method further comprises administering to said subject one or more additional effective amounts of the one or more (e.g., one or two, e.g., one) possible stereoisomers of (2-(4-methoxy)quinolin-4-yl)(piperidin-2-yl)methanol (e.g., S,R stereoisomer, sometimes referred to as NSC 23925b or compound 11), or pharmaceutically acceptable salt thereof, after administration of said subsequent cancer treatment.

In some implementations, the cancer treatment is selected from the group consisting of surgery, chemotherapy, radiation therapy, immunotherapy and monoclonal antibody therapy.

In some implementations of the compositions and methods described herein, the compound is NSC 23925b.

Implementations can include one or more of the following features.

NSC 23925b is free (e.g., substantially free) of its enantiomer

NSC 23925b is free (e.g., substantially free) of one or both of its corresponding diastereomers.

NSC 23925b is free (e.g., substantially free) of its enantiomer and is free (e.g., substantially free) of one or both of its corresponding diastereomers.

### DEFINITIONS

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Compounds of the present disclosure can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, inorganic, mineral or organic acid salts of basic residues such as amines; alkali or organic salts of acidic residues such as carboxylic acids; and the like. The pharmaceutically acceptable salts of the present disclosure include the conventional non-toxic salts or the quaternary ammonium salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Suitable pharmaceutically acceptable salts of the compounds described herein can include acid addition salts which may, for example, be formed by mixing a solution of the compound with a solution of a pharmaceutically acceptable acid, such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. In addition, lists of suitable salts are found in Remington's Pharmaceutical Sciences, 20th ed., Lippincott Williams & Wilkins, 2003, p. 2100 and Journal of Pharmaceutical Science, 66, 2 (1977), each of which is incorporated herein by reference in its entirety.

The term “pharmaceutically acceptable acid” refers to inorganic or organic acids that exhibit no substantial toxicity. Examples of pharmaceutically acceptable acids include, but are not limited to hydrochloric acid, hydrobromic acid, sulfuric acid, nitric acid, phenylsulfonic acid, methanesulfonic acid, fumaric acid, maleic acid, succinic acid, benzoic acid, acetic acid, citric acid, tartaric acid, phosphoric acid, carbonic acid, and the like.

A “protecting group” as used herein is a derivative of a chemical functional group which would otherwise be incompatible with the conditions required to perform a particular reaction which, after the reaction has been carried out, can be removed to re-generate the original functional group, which is thereby considered to have been “protected”. Any chemical functionality that is a structural component of any of the reagents used to synthesize compounds described herein may be optionally protected with a chemical protecting group if such a protecting group is useful in the synthesis of compounds described herein. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis by Theodora W. Greene, Peter G. M. Wuts, John Wiley & Sons Ltd., the entire disclosure of which is incorporated herein by reference. Examples of protecting groups include acyl groups, allyl groups, benzyl groups, trityl groups, alkyl-, allyl-, or aryloxycarbonyl groups, alkyl-, allyl, or arylcarbamoyl groups, trialkylsilyl groups, and alkly- or arylsulfonyl groups.

As used herein, “substantially diastereomerically pure” refers to compositions containing at least 55% (e.g., at least: 60%; 70%; 80%; 85%; 90%; 95%; 97%; 98%; 99%; 99.5%; 99.8%; or 99.9%) of a single diastereomer, relative to the total amount of all the diastereomers in the composition.

As used herein, the “optical rotation” of a chemical compound [α] refers to the observed angle of optical rotation a when plane-polarized light is passed through a sample of a compound as described herein with a path length of 1 decimeter and a sample concentration of 1 gram per 1 milliliter. Optical rotation values are reported using degrees. As used herein, the term “about” is +/−1%, +/−5%, or +/−10% of the recited value.

As used herein, the terms “(R)” or “R” and “(S)” or “S” are based on naming conventions well known to one of skill in the art, for example, an R-configuration is based on a compound's actual geometry, typically using the Cahn-Ingold-Prelog priority rules to classify the form (Smith M. B., March, J, March's Advanced Organic Chemistry, 5^{th} ed. Wiley-Interscience, NY, 2001, p 139-141). As described herein, compounds of the present disclosure have been characterized as having R configuration or an S configuration.

The term “subject” is used throughout the specification to describe an animal, human or non-human, to whom treatment and/or imaging according to the methods of the present disclosure is provided. Veterinary and non-veterinary applications are contemplated. The term includes but is not limited to mammals, e.g., humans, other primates, pigs, rodents such as mice and rats, rabbits, guinea pigs, hamsters, cows, horses, cats, dogs, sheep and goats.

### Synthesis

The novel compounds of the present disclosure can be prepared in a variety of ways known to one skilled in the art of organic synthesis. The compounds described herein can be synthesized using the methods as hereinafter described below, together with synthetic methods known in the art of synthetic organic chemistry or variations thereon as appreciated by those skilled in the art.

The compounds described herein can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds described herein were prepared as illustrated in Scheme 1. Reaction of commercially available indolin-2,3-dione with 1-(4-methoxyphenyl)ethanone under basic conditions provided quinoline carboxylic acid 1. Subsequent treatment of 1 with methanol and sulfuric acid yielded quinoline methyl ester 2 which underwent 1,2-addition with pyridin-2-yllithium to afford ketone 3. Reduction of ketone 3 with a hydride reducing agent, such as sodium borohydride, provided a racemic mixture of pyridine alcohol 4. Hydrogenation of the pyridine of 4 in the presence of a catalyst such as Adams' catalyst afforded NSC23925.

Attempts to separate the diastereomers of NSC23925 using preparative HPLC and chiral HPLC proved difficult. Therefore, NSC23925 was converted to a carbamate functionality such as t-butoxycarbonyl (t-Boc) to facilitate the chiral separation. NSC23925 was Boc-protected employing triethylamine (TEA) and t-Boc-anhydride to provide a mixture of carbamates, which were then separated by column chromatography into the erythro and threo diastereomeric pairs 6 and 7, respectively. Chiral separation of 6 and 7, followed by removal of the t-Boc protecting group under acidic conditions afforded the substantially pure stereoisomers NSC23925a, NSC23925b, NSC23925c, and NSC23925d.

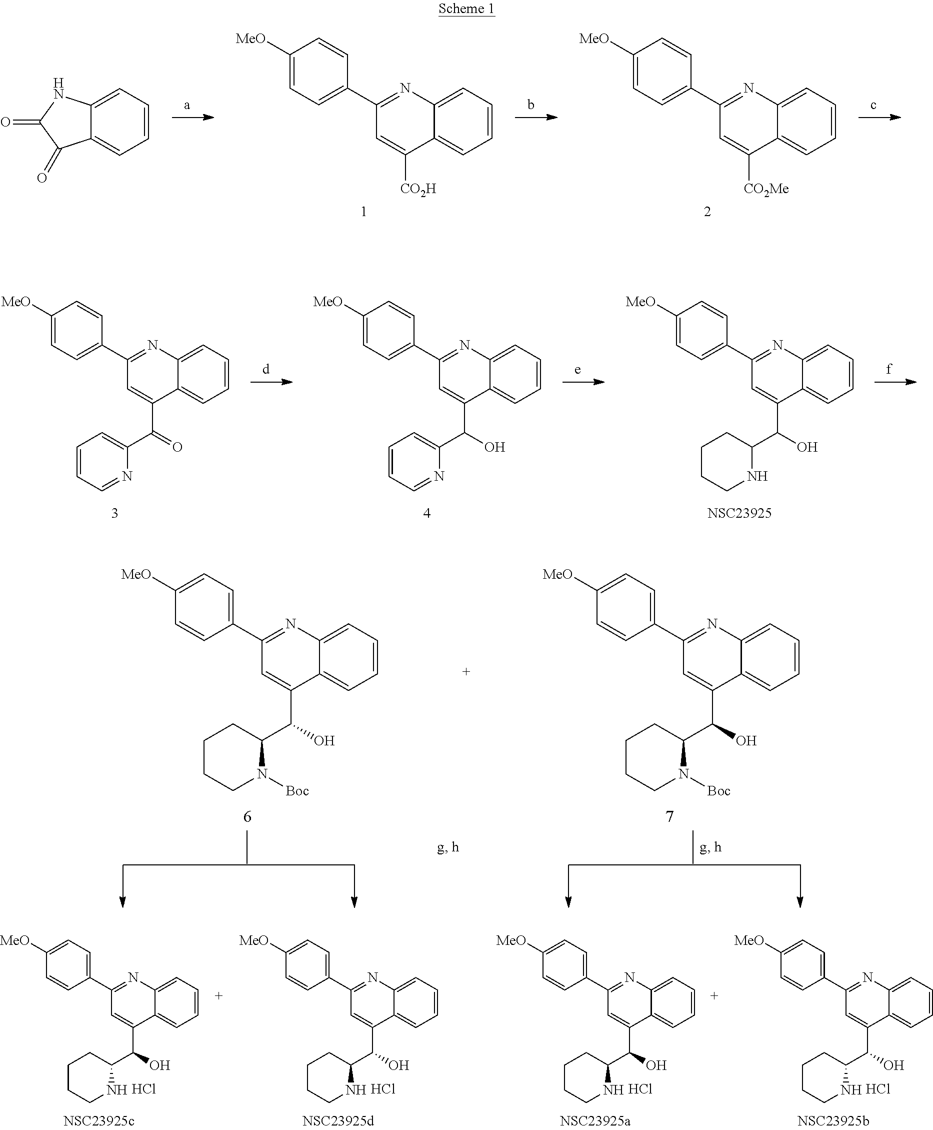

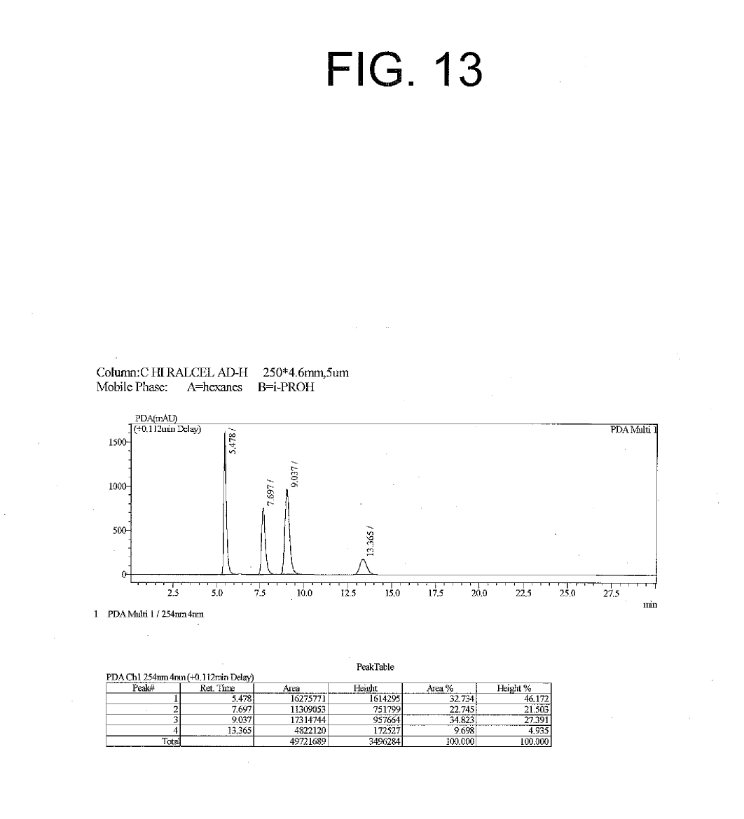

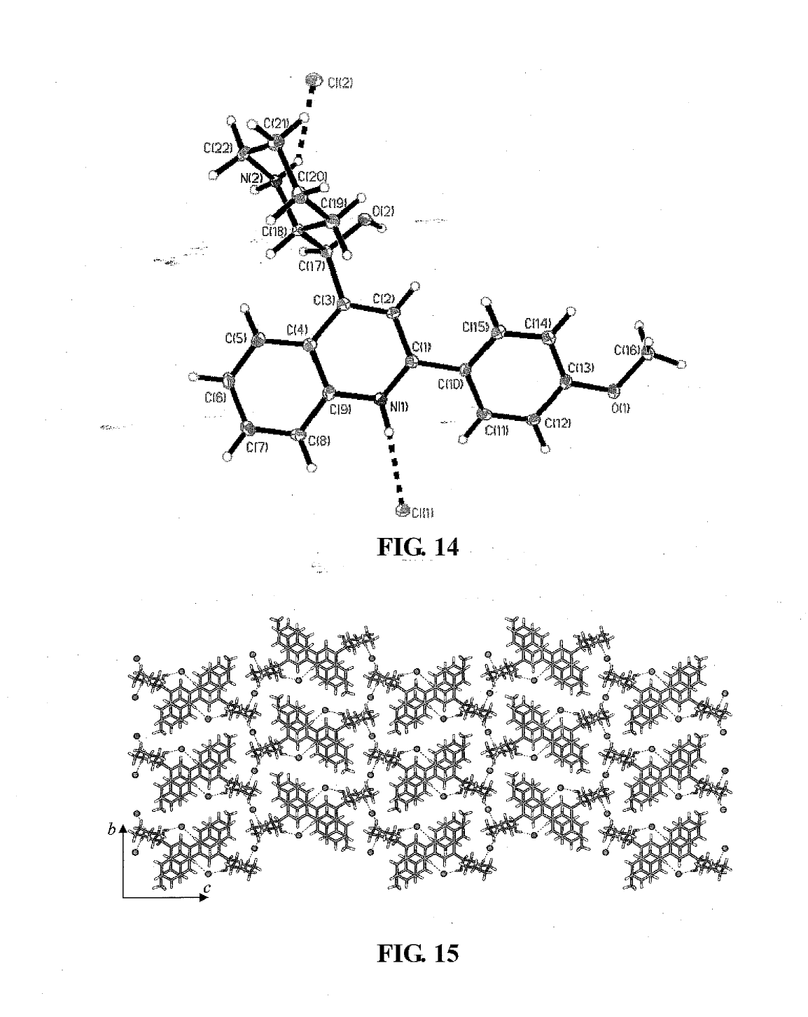

The absolute stereochemistry of NSC23925b was determined using both ¹H NMR and x-ray crystallography. The erythro pair (NSC23925a and NSC23925b) could be distinguished from the threo pair using ¹H NMR; specifically by examination of the amino alcohol C—H proton coupling constants and applying the observation of Solladie-Cavallo et al. (J. Org. Chem. 2003, 68, 7308) that ³J-erythro<³J-threo for amino alcohols having similar CH(OH)—CH(NH) system. The absolute stereochemistry of NSC23925b was determined using x-ray crystallography (see FIGS. 14 and 15 and Examples section) after separation, purification, and crystallization of NSC23925b using the methods described herein. The methods used to crystallize NSC23925b are described in the Examples section.

### Methods of Use

The present disclosure provides a method for using a compound described herein and compositions thereof, to reduce (the incidence or severity of) drug resistance in a subject undergoing cancer treatment. Compounds described herein can be utilized to reduce (e.g. reverse) drug resistance in cancer cells. In some embodiments, the compounds described herein reduce (e.g. reverses) drug resistance through inhibiting the function of Pgp.

The method comprises administering to a subject in need thereof an effective amount of a compound described herein, thereby reducing drug resistance in the subject undergoing cancer treatment. The effective amount of a compound described herein is an amount sufficient to reduce (the incidence or severity of) drug resistance in the subject. An effective amount of a compound described herein can be provided in one or a series of administrations (or doses). The effective amount of a compound described herein is generally determined by the physician on a case-by-case basis and is within the skill of one skilled in the art.

In some embodiments, the compounds described herein are administered to the subject at a dose that is lower than the dose required to produce cytotoxicity in the subject. In some embodiments, the compounds described herein are administered to the subject at a dose at least 10 fold lower than what is required to produce cytotoxicity in the subject. In some embodiments, the compounds described herein administered at a dose at least 50 fold lower than that is required to produce cytotoxicity in the subject being treated.

In another aspect, the present disclosure provides for a method for treating cancer in a subject by using the compounds described herein. The method includes a) identifying a subject undergoing cancer treatment and having developed or being susceptible to developing drug resistance; and b) administering to the subject an effective amount of a compound as described herein, wherein the effective amount is sufficient to reduce drug resistance in the subject; and the method thereby treats cancer in a subject. Such an effective amount may be an amount that is therapeutically and/or prophylactically effective.

Determination of a therapeutically effective amount or a prophylactically effective amount of a compound as described herein, can be readily made by the physician or veterinarian (the “attending clinician”), as one skilled in the art, by the use of known techniques and by observing results obtained under analogous circumstances. The amount (or dose) may be varied depending upon the requirements of the subject in the judgment of the attending clinician; the severity of the condition being treated and the particular compound being employed. In determining the effective amount for the purpose of the present disclosure, a number of factors are considered by the attending clinician, including, but not limited to: the age, sex and weight of the subject being treated; the specific cancer involved; pharmacodynamic characteristics of the particular anti-cancer agent used and its mode and route of administration; the desired time course of treatment; the species of mammal; its size, age, and general health; the degree of or involvement or the severity of the cancer; the response of the individual subject; the degree or severity of the drug resistance symptom in the subject; the mode of administration; the bioavailability characteristics of the preparation administered; the dose regimen selected; the kind of concurrent treatment (i.e., the interaction of the compounds described herein with other co-administered therapeutics); and other relevant circumstances.

Further, the identification of those subjects undergoing cancer treatment and having developed or being susceptible to developing drug resistance is well within the ability and knowledge of one skilled in the art. Certain of the methods for identification of subjects which are at risk of developing drug resistance which can be treated by the subject method are appreciated in the medical arts, such as prior medical history, family history, and the presence of risk factors associated with the development of the phenomenon in the subject. A clinician skilled in the art can readily identify such candidate subjects, by the use of, for example, clinical tests, physical examination and medical/family history screen.

While using a compound described herein to treat cancer in a subject, the methods sometimes involve discontinuation of the cancer treatment which the subject has undergone or is undergoing. In some embodiments, the subject in need is then administered a subsequent cancer treatment after the aforementioned cancer treatment has been discontinued and a compound as described herein has been administered. This subsequent cancer treatment can be the same as, or different from, the discontinued cancer treatment.

In some embodiments, an additional effective amount of a compound as described herein is administered to the subject after the administration of the subsequent cancer treatment. In some embodiments, the methods described herein can include repeatedly administering to the subject in need of treatment an additional effective amount of a compound as described herein after administration of the subsequent cancer treatment.

An attending physician or veterinarian can readily make a determination on when to administer the additional effective amount of the compound described herein after the administration of the subsequent cancer treatment. Such determination can be made based on subjective and/or objective standards. For example, an additional effective amount of a compound described herein is administered between within about an hour to about 6 months after the subsequent cancer treatment has been introduced to treat the subject. Other examples can include the administration of the additional effective amount of the compound described herein 1 day, 1 week, 2 weeks, one month, or six months after the administration of the subsequent cancer treatment. The extent or severity of the drug resistance symptoms may be determined periodically throughout treatment. For example, the extent or severity of the drug resistance symptoms may be checked every few hours, days or weeks to assess the efficacy of the treatment. A decrease in extent or severity of the drug resistance symptoms indicates that the treatment is efficacious. The method described may be used to screen or select subjects that may benefit from treatment with a compound as described herein.

The cancer treatment includes, but is not limited to, surgery, chemotherapy, radiation therapy, immunotherapy or monoclonal antibody therapy.

A method of assessing the efficacy of the subject method to treat cancer in a subject includes determining the pre-treatment extent of a cancer by methods well known in the art and then administering to the subject an effective amount of a compound described herein. After an appropriate period of time after the administration of the subject method (e.g., 1 day, 1 week, 2 weeks, one month, six months), the extent/severity of the drug resistance is determined again. The modulation (e.g., decrease) of the extent or severity of the drug resistance in the subject indicates efficacy of the treatment. The extent or severity of the drug resistance may be determined periodically throughout treatment. For example, the extent or severity of the drug resistance may be checked every few hours, days or weeks to assess the further efficacy of the treatment. Further, a decrease in extent or severity of the cancer also indicates that the treatment is efficacious. The method described may be used to screen or select subjects that may benefit from treatment with a compound as described herein.

Cancers, for the purpose of the present disclosure, include a class of diseases in which a group of cells display uncontrolled growth, invasion, and/or metastasis. Cancer, as mentioned herein, may be, but is not limited to, e.g., a cancer of breast, respiratory tract, brain, reproductive organs, digestive tract, urinary tract, eye, liver, skin, bone, head and neck, thyroid, parathyroid or a distant metastasis of a solid tumor. In some embodiments, cancers can also include lymphomas, sarcomas, and leukemia.

Examples of breast cancer include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalamic glioma, cerebellar and cerebral astrocytoma, medulloblastoma, ependymoma, as well as neuroectodermal and pineal tumor.

Cancers of the male reproductive organs include, but are not limited to, prostate and testicular cancer. Cancers of the female reproductive organs include, but are not limited to, endometrial, cervical, ovarian, vaginal, and vulvar cancer, as well as sarcoma of the uterus.

Cancers of the digestive tract include, but are not limited to, anal, colon, colorectal, esophageal, gallbladder, gastric, pancreatic, rectal, small-intestine, and salivary gland cancers.

Cancers of the urinary tract include, but are not limited to, bladder, penile, kidney, renal pelvis, ureter, urethral and human papillary renal cancers.

Eye cancers include, but are not limited to intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Examples of bone cancers include, but are not limited to, osteosarcoma, Ewing's sarcoma, malignant fibrous histiocytoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These diseases or disorders have been well characterized in humans, but also exist with a similar etiology in other mammals.

Another aspect of the present disclosure is to use a compound described herein in the treatment or prevention in a subject of a disease, disorder or symptom thereof as described herein.

### Combination Therapy

A compound of the present disclosure can be used alone or in combination with an additional therapeutic agent to treat such diseases described herein. Determination on such an additional therapeutic agent suitable to be combined with a compound of the present disclosure can be readily made by the skilled artisan. For example, the additional agent can be a therapeutic agent art-recognized as being useful to treat the disease or condition being treated by the compound of the present disclosure. The additional agent also can be an agent that imparts a beneficial attribute to the therapeutic composition e.g., an agent that affects the viscosity of the composition.

It should further be understood that the combinations encompassed by the present disclosure are those combinations useful for their intended purpose. The agents set forth below are illustrative for purposes and not intended to be limited. The combinations, which are part of the present disclosure, can be the compounds of the present disclosure and at least one additional agent selected from the lists below. The combination can also include more than one additional agent, e.g., two or three additional agents if the combination is such that the formed composition can perform its intended function.

In some embodiments, the additional agent is an anti-cancer therapeutic agent. Such an anti-cancer therapeutic agent and a compound of the present disclosure can be administered simultaneously. Alternatively, the compound of the present disclosure and the anti-cancer therapeutic agent can be administered to the subject sequentially. In some embodiments, the compound of the present disclosure is administered to the subject prior to the administration of the anti-cancer agent. In some embodiments, the compound of the present disclosure is administered to the subject after the administration of the anti-cancer agent.

The anti-cancer therapeutic agent can be, but is not limited to, asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, or zalypsis.

Proteosome inhibitors (e.g., MG-132), hydroxyureas (e.g., Hydrea or hydroxycarbamide) or kinase inhibitors (e.g., GLEEVEC) can also be used in combination with the compounds described herein. Certain therapeutic monoclonal antibodies can also be used with a compound described herein. Such monoclonal antibodies include, but not limited to, alemtuzumab, bevacizumab, cetuximab, efalizumab, ibritumomab tiuxetan, 111in-capromab, imciromab, panitumumab, gemtuzumab ozogamicin, rituximab, tositumomab, and trastuzumab.

In some embodiments, the anti-cancer therapeutic agent is paclitaxel, doxorubicin, docetaxel, calcein-AM, daunorubicin, gemcitabine, rhodamine-123, ET-743, vincristin or zalypsis. Separately, the anti-cancer therapeutic agent may be a cytotoxic drug selected from the group consisting of anthracyclines, vinca alkaloids and taxanes.

Non-limiting examples of other therapeutic agents that can be used in combination can include, e.g., antiangiogenesis agents, anti-proliferative agents, DNA-RNA transcription regulators, DNA synthesis inhibitors, enzyme inhibitors/activators, HSP-90 inhibitors, microtubule inhibitors, gene regulators, antibodies, etc. A list of the additional therapeutic agents that can be used in combination with a compound of the present disclosure can be found in Harrison's Principles of Internal Medicine, 17th Edition, Eds. T. R. Harrison et al. McGraw-Hill N.Y., NY; and the Physicians' Desk Reference 62nd Edition 2008, Oradell N.J., Medical Economics Co., the contents of which are incorporated by reference in its entirety.

Examples of antiangiogenesis agents include, but are not limited to, angiostatin k1-3 human, dl-α-difluoromethylornithine hydrate hydrochloride, endostatin, endostatin murine, fumagillin, genistein, minocycline hydrochloride, silibinin, silymarin, staurosporine, su 5416, and thalidomide.

Examples of anti-proliferative agents include, but are not limited to, 25-hydroxycholesterol, 7β-hydroxycholesterol, aloe-emodin, apigenin, berberine, caffeine, dichloro-methylenediphosphonic acid disodium salt, emodin, HA 14-1, hyperforin, N-acetyl-d-sphingosine, N-hexanoyl-d-sphingosine, parthenolide, β-ionone, and trans-cinnamaldehyde.

Examples of DNA-RNA transcription regulators include, but are not limited to, 5,6-dichlorobenzimidazole, 1-β-d-ribofuranoside, actinomycin d, homoharringtonine, and idarubicin hydrochloride.

Examples of DNA synthesis inhibitors include, but are not limited to, amethopterin, 2-fluoroadenine-9-β-D-arabinofuranoside, 5-fluoro-5′-deoxyuridine, 6-mercaptopurine, 6-thioguanine, aminopterin, cytosine-β-D-arabinofuranoside, ganciclovir, and hydroxyurea.

Examples of enzyme activators/inhibitors include, but are not limited to, forskolin, (−)-deguelin, (−)-depudecin, camptothecin, 2-imino-1-imidazolidineacetic acid, 2-propylpentanoic acid, 7-ethyl-10-hydroxycamptothecin, DL-aminoglutethimide, apicidin and etoposide.

Examples of HSP-90 inhibitors include, but are not limited to, 17-(allylamino)-17-demethoxygeldanamycin, geldanamycin, and rifabutin.

Examples of microtubule inhibitors include, but are not limited to, colchicine, dolastatin, nocodazole, podophyllotoxin, rhizoxin, vinblastine, and vinorelbine hydrate ditartrate salt.

Examples of gene regulators, include, but are not limited to, 13-cis-Retinoic acid, 4-Hydroxytamoxifen, 5-Aza-2′-deoxycytidine, 5-Azacytidine, 9-cis-Retinoic acid, Cholecalciferol, ciglitizone, cyproterone acetate, epitestosterone, flutamide, GW9662, glycyrrhizic acid ammonium salt, melatonin, mifepristone, procainamide hydrochloride, raloxifene, retinoic acid, retinol, tamoxifen, tetradecylthioacetic acid, and troglitazone.

Examples of antibodies include, but not limited to, alemtuzumab, bevacizumab, cetuximab, efalizumab, ibritumomab tiuxetan, 111in-capromab, imciromab, panitumumab, gemtuzumab ozogamicin, rituximab, tositumomab, and trastuzumab.

The compounds of the present disclosure and the additional therapeutic agent(s) can be administered to the subject in the same pharmaceutical composition or in different pharmaceutical compositions (at the same time or at different times).

In another aspect, the present disclosure provides for the use of a compound of the present disclosure, alone or together with one or more additional therapeutic agents in the manufacture of a medicament, either as a single composition or as separate dosage forms, for treatment or prevention in a subject of a disease, disorder or symptom set forth herein.

### Dosages

Suitable dosages of a compound of the present disclosure can be empirically determined by an administering physician. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein.

In some embodiments, a dose of a compound of the present disclosure varies from about 0.001 mg/kg of body weight to about 100 mg/kg. Treatment can be initiated with smaller dosages. The dosage may then be increased by small increments until the optimum effect under the circumstances is reached. For convenience, the total dosage can be divided and administered in portions during the administration period if desired.

The dosages can be varied depending upon the requirements of the subject in the judgment of the attending clinician; the severity of the condition being treated and the particular compound being employed. In determining the effective amount or dose, and the prophylactically effective amount or dose, a number of factors are considered by the attending clinician, including, but are not limited to: the age, sex and weight of the subject being treated; the specific cancer involved; pharmacodynamic characteristics of the particular anti-cancer agent used and its mode and route of administration; the desired time course of treatment; the species of mammal; its size, age, and general health; the degree of or involvement or the severity of the cancer; the response of the individual subject; the degree or severity of the drug resistance symptom in the subject; the mode of administration; the bioavailability characteristics of the preparation administered; the dose regimen selected; the kind of concurrent treatment (i.e., the interaction of a compound described herein with other co-administered therapeutics); and other relevant circumstances.

The dosage of a compound described herein can vary from about 0.01 mg to about 5,000 mg per day. In some instances, the dosage varies from about 100 mg to about 4000 mg per day, or about 1000 mg to about 3000 mg per day. Ascertaining dosage ranges is well within the skill of one in the art. In some embodiments, the dosage of a compound described herein can range from about 0.001 to about 100 mg/kg of body weight. For example, ranges can be about 0.01 to about 30 mg/kg body weight, about 0.1 to 20 mg/kg body weight, about 1 to 10 mg/kg, about 2 to 9 mg/kg, about 3 to 8 mg/kg, about 4 to 7 mg/kg, or about 5 to 6 mg/kg body weight. Such dosages can vary, for example, depending on whether multiple administrations are given, tissue type and route of administration, the condition of the individual, the desired objective and other factors known to those of skill in the art. Administrations can be conducted frequently, for example, on a regular daily or weekly basis, until a desired, measurable parameter is detected, such as diminution of disease symptoms. Administration can then be diminished, such as to a biweekly or monthly basis

### Formulations and Dosage Forms

The present disclosure provides pharmaceutical compositions containing an effective amount of a compound described herein. The pharmaceutical compositions can also include a pharmaceutically acceptable carrier or diluent. These compositions can be utilized to achieve the desired pharmacological effect by administration to a subject in need thereof.

In some embodiments, the effective amount of a compound described herein and included in the pharmaceutical composition is at a dose lower than that required to produce cytotoxicity in the subject. In some embodiments, the effective amount of a compound described herein is at a dose at least 10 fold lower than that is required to produce cytotoxicity. The effective amount is effective to reduce the severity or incidence of dug resistance in a subject undergoing cancer treatment, as described previously.

A pharmaceutically acceptable carrier is preferably a carrier that is relatively non-toxic and innocuous to a subject at concentrations consistent with effective activity of the active ingredient so that any side effects ascribable to the carrier do not vitiate the beneficial effects of the active ingredient. An effective amount of a compound is preferably that amount which reduces drug resistance in the subject undergoing cancer treatment. The compounds described herein can be administered with pharmaceutically-acceptable carriers well known in the art using any effective conventional dosage unit forms, including immediate, slow and timed release preparations, orally, parenterally, topically, nasally, ophthalmically, optically, sublingually, rectally, or vaginally.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In some embodiments, a compound described herein may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of the present disclosure may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

Compounds as described herein may also be administered parenterally, in other words, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of the present disclosure are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

Parenteral compositions of the present disclosure will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions can be in the form of sterile injectable aqueous suspensions. Such suspensions can be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Compositions of the present disclosure may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present disclosure employs transdermal delivery devices (“patches”). Such transdermal patches can be used to provide continuous or discontinuous infusion of the compounds of the present disclosure in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches can be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the present disclosure can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference in its entirety: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

Acidifying agents (examples include but are not limited to acetic acid, citric acid, fumaric acid, hydrochloric acid, nitric acid);

Alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

Adsorbents (examples include but are not limited to powdered cellulose and activated charcoal);

Aerosol propellants (examples include but are not limited to carbon dioxide, CCl₂F₂, F₂ClC—CClF₂ and CClF₃)

Air displacement agents (examples include but are not limited to nitrogen and argon);

Antifungal preservatives (examples include but are not limited to benzoic acid, butylparaben, ethylparaben, methylparaben, propylparaben, sodium benzoate);

Antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

Antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

Binding materials (examples include but are not limited to block polymers, natural and synthetic rubber, polyacrylates, polyurethanes, silicones, polysiloxanes and styrene-butadiene copolymers);

Buffering agents (examples include but are not limited to potassium metaphosphate, dipotassium phosphate, sodium acetate, sodium citrate anhydrous and sodium citrate dihydrate);

Carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

Chelating agents (examples include, but are not limited to, edetate disodium and edetic acid)

Colorants (examples include, but are not limited, to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

Clarifying agents (examples include, but are not limited to, bentonite);

Emulsifying agents (examples include, but are not limited to, acacia, cetomacrogol, cetyl alcohol, glyceryl monostearate, lecithin, sorbitan monooleate, polyoxyethylene 50 monostearate);

Encapsulating agents (examples include, but are not limited to, gelatin and cellulose acetate phthalate);

Flavorants (examples include, but are not limited to, anise oil, cinnamon oil, cocoa, menthol, orange oil, peppermint oil and vanillin);

Humectants (examples include, but are not limited to, glycerol, propylene glycol and sorbitol);

Levigating agents (examples include, but are not limited to, mineral oil and glycerin);

Oils (examples include, but are not limited to, arachis oil, mineral oil, olive oil, peanut oil, sesame oil and vegetable oil);

Ointment bases (examples include, but are not limited to, lanolin, hydrophilic ointment, polyethylene glycol ointment, petrolatum, hydrophilic petrolatum, white ointment, yellow ointment, and rose water ointment);

Penetration enhancers (transdermal delivery) (examples include, but are not limited to, monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);

Plasticizers (examples include but are not limited to diethyl phthalate and glycerol);

Solvents (examples include, but are not limited to, ethanol, corn oil, cottonseed oil, glycerol, isopropanol, mineral oil, oleic acid, peanut oil, purified water, water for injection, sterile water for injection and sterile water for irrigation);

Stiffening Agents (examples include, but are not limited to, cetyl alcohol, cetyl esters wax, microcrystalline wax, paraffin, stearyl alcohol, white wax and yellow wax);

Suppository Bases (examples include, but are not limited to, cocoa butter and polyethylene glycols (mixtures));

Surfactants (examples include, but are not limited to, benzalkonium chloride, nonoxynol 10, oxtoxynol 9, polysorbate 80, sodium lauryl sulfate and sorbitan mono-palmitate);

Suspending Agents (examples include, but are not limited to, agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

Sweetening Agents (examples include, but are not limited to, aspartame, dextrose, glycerol, mannitol, propylene glycol, saccharin sodium, sorbitol and sucrose);

Tablet anti-adherents (examples include, but are not limited to, magnesium stearate and talc);

Tablet binders (examples include, but are not limited to, acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

Tablet and capsule diluents (examples include, but are not limited to, dibasic calcium phosphate, kaolin, lactose, mannitol, microcrystalline cellulose, powdered cellulose, precipitated calcium carbonate, sodium carbonate, sodium phosphate, sorbitol and starch);

Tablet coating agents (examples include, but are not limited to, liquid glucose, hydroxyethyl cellulose, hydroxypropyl cellulose, hydroxypropyl methylcellulose, methylcellulose, ethylcellulose, cellulose acetate phthalate and shellac);

Tablet direct compression excipients (examples include, but are not limited to, dibasic calcium phosphate);

Tablet disintegrants (examples include, but are not limited to, alginic acid, carboxymethylcellulose calcium, microcrystalline cellulose, polacrillin potassium, crosslinked polyvinylpyrrolidone, sodium alginate, sodium starch glycollate and starch);

Tablet glidants (examples include, but are not limited to, colloidal silica, corn starch and talc);

Tablet lubricants (examples include, but are not limited to, calcium stearate, magnesium stearate, mineral oil, stearic acid and zinc stearate);

Tablet/capsule opaquants (examples include, but are not limited to, titanium dioxide);

Tablet polishing agents (examples include, but are not limited to, carnuba wax and white wax);

Thickening agents (examples include, but are not limited to, beeswax, cetyl alcohol and paraffin);

Tonicity agents (examples include, but are not limited to, dextrose and sodium chloride);

Viscosity increasing agents (examples include, but are not limited to, alginic acid, bentonite, carbomers, carboxymethylcellulose sodium, methylcellulose, polyvinyl pyrrolidone, sodium alginate and tragacanth); and

Wetting agents (examples include, but are not limited to, heptadecaethylene oxycetanol, lecithins, sorbitol monooleate, polyoxyethylene sorbitol monooleate, and polyoxyethylene stearate).

Pharmaceutical compositions according to the present disclosure can be illustrated as follows:

Sterile IV Solution:

A 5 mg/mL solution of a compound described herein can be made using sterile, injectable water, and the pH is adjusted if necessary. The solution is diluted for administration to 1-2 mg/mL with sterile 5% dextrose and is administered as an IV infusion over about 60 minutes.

Lyophilized Powder for IV Administration:

A sterile preparation can be prepared with (i) 100-1000 mg of a compound described herein as a lyophilized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular Suspension:

The following solution or suspension can be prepared, for intramuscular injection:

50 mg/mL of a compound described herein

5 mg/mL sodium carboxymethylcellulose

4 mg/mL TWEEN 80

9 mg/mL sodium chloride

9 mg/mL benzyl alcohol

Hard Shell Capsules:

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules:

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets:

A large number of tablets are prepared by conventional procedures so that the dosage unit is 100 mg of active ingredient, 0.2 mg of colloidal silicon dioxide, 5 mg of magnesium stearate, 275 mg of microcrystalline cellulose, 11 mg of starch, and 98.8 mg of lactose. Appropriate aqueous and non-aqueous coatings may be applied to increase palatability, improve elegance and stability or delay absorption.

Immediate Release Tablets/Capsules:

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

The pharmaceutical compositions of present disclosure can further include additional therapeutic agent as previously discussed. In some embodiments, the additional therapeutic agent is an anti-cancer therapeutic agent. Such an anti-cancer agent can be, for example, asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, or zalypsis. In some embodiments, the additional therapeutic agent is paclitaxel, doxorubicin, docetaxel, calcein-AM, daunorubicin, gemcitabine, rhodamine-123, ET-743, vincristin or zalypsis.

When a compound of the present disclosure is administered as pharmaceuticals, to humans and animals, it can be given per se or as a pharmaceutical composition containing, for example, 0.1 to 99.5% (or 0.5 to 90%) of active ingredient in combination with a pharmaceutically-acceptable carrier.

Regardless of the route of administration, the compound of the present disclosure, which can be used in a suitable hydrated form, and/or the pharmaceutical compositions, are formulated into pharmaceutically-acceptable dosage forms by conventional methods known to those of skill in the art.

Actual dosage levels and time course of administration of the active ingredients in the pharmaceutical compositions of the present disclosure can be varied so as to obtain an amount of the active ingredient which is effective to achieve the desired therapeutic response for a particular subject, composition, and mode of administration, without being toxic to the subject. In some embodiments, the dose range is from about 0.001 to about 100 mg/kg per day. Other examples for the dose range are discussed supra. In another embodiment, the dose of a compound described herein is the maximum that a subject can tolerate and not develop serious side effects.

### VII. Kits

The present disclosure also provides kits for treating diseases delineated herein. A typical kit of the present disclosure includes a compound, a pharmaceutical formulation or a combination described in this document, and instructions for use. The instructions for use may include information on dosage, method of delivery, storage of the kit, etc. In some embodiments, the kit includes instructions for administering the compound, formulation or combination of the present disclosure.

A kit can include instructions and/or information for identification of a subject in need for treatment. In certain embodiments, the kit may include instructions to treat a subject suffering from or susceptible to drug resistance in a cancer treatment. Sometimes, a kit may include instructions to treat or prevent cancer.

In some embodiments, the kit further includes an additional therapeutic agent as previously discussed. In some embodiments, the additional therapeutic agent is an anti-cancer therapeutic agent as discussed supra.

The kits can also include reagents, for example, test compounds, buffers, media (e.g., cell growth media), cells, etc. Test compounds can include known compounds or newly discovered compounds, for example, combinatorial libraries of compounds.

Kits of the present disclosure can further comprise devices that are used to administer a compound described herein. Examples of such devices include, but are not limited to, intravenous cannulation devices, syringes, drip bags, patches, topical gels, pumps, containers that provide protection from photodegradation, autoinjectors, and inhalers.

Kits of the present disclosure can also comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Without wishing to be bound by any theory, a compound identified through the methods described herein is capable of reducing or modulating drug resistance in a subject undergoing cancer treatment. In some embodiments, the compound identified through the methods described herein demonstrates specificity in modulating or reducing drug resistance in cancer cell lines of the subject being treated.

### EXAMPLES

The invention is further described in the following examples, which do not limit the scope of the invention described in the claims.

### Example 1

### Synthesis of the NSC23929 Isomers

### General Methods

All evaporations were carried out in vacuo with a rotary evaporator. Analytical samples were dried in vacuo (1-5 mmHg) at room temperature. Thin layer chromatography (TLC) was performed on silica gel plates with fluorescent indicator. Spots were visualized by UV light (214 and 254 nm). Purification by column and flash chromatography was carried out using silica gel (300-400 mesh). Solvent systems are reported as volume percent mixture. All NMR spectra were recorded on a Bruker WH-300 (300 MHz) spectrometer. ¹H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

LCMS spectra were obtained on an Agilent 1200 series 6110 or 6120 mass spectrometer with electrospray ionization. LC/MS: Method 1 (5-100a & 5-100b)=(Waters X Bridge C18, 50 mm*4.6 mm*3.5 um, 2.3 min gradient, 5% [0.05% TFA/CH3CN]: 95%[0.05% TFA/H2O] to 100% [0.05% TFA/CH3CN] for 1.6 min and hold 100% [0.05% TFA/CH3CN] for 0.7 min; Method 2 (5-100a (test) and 5-100b (test))=(Waters X Bridge C18, 50 mm*4.6 mm*3.5 um, 3.5 min gradient, 5% [0.05% TFA/CH3CN]: 95%[0.05% TFA/H2O] to 100% [0.05% TFA/CH3CN] for 1.6 min and hold 100% [0.05% TFA/CH3CN] for 1.9 min; Method 3 (5-100A 5)=(Waters X Bridge C18, 50 mm*4.6 mm*3.5 um, 5.8 min gradient, 5% [0.05% TFA/CH3CN]: 95% [0.05% TFA/H2O] to 100% [0.05% TFA/CH3CN] for 5.0 min and hold 100% [0.05% TFA/CH3CN] for 0.8 min.

Preparative chiral HPLC purification was performed on a Shimadzu LC 20 with UV detector SPD-20A at 35° C. using CHIRALPAK IA (0.46 cm I.D.×25 cm L.) and using Hexane/Ispropanol/DEA=70/30/0.1 (V/V/V) as the mobile phase. Flow rate was 1.0 mL/min (all solvents were HPLC grade). The HPLC system was monitored at 254 nm.

Analytical chiral HPLC spectra were run on a Shimadzu LC 20 with UV detector SPD-20A at 35° C. using Chiralpak AD-H (0.46×25 cm) and using Hexane/Ispropanol/DEA=70/30/0.1 (V/V/V) during 20 min as the mobile phase. Flow rate was 1.0 mL/min (all solvents were HPLC grade). The HPLC system was monitored at 254 nm. Optical rotation was run on a Polarimeter Perkin Elmer Model 341 at 20° C.

Optical Rotation Determination:

### NSC23925b (0.26 g, Peak-2)):

16 mg of sample dissolved in 10 mL of MeOH in 10 ml volumetric flask.
Then the optical rotation was measured by automatic digital polarimeter 2-3 times and finally took the average data.

### NSC23925c (0.6 g, Peak-1)):

20 mg of sample dissolved in 10 mL of MeOH in 10 ml volumetric flask.
Then the optical rotation was measured by automatic digital polarimeter 2-3 times and finally take the average data.

### NSC23925d (0.6 g, Peak-2):

20 mg of sample dissolved in 10 mL of MeOH in 10 ml volumetric flask.
Then the optical rotation was measured by automatic digital polarimeter 2-3 times and finally take the average data.

### Synthesis

### 2-(4-Methoxyphenyl)quinoline-4-carboxylic acid (1)

Indoline-2,3-dione (30.6 g, 203.9 mmol), 1-(4-methoxyphenyl)ethanone (25.0 g, 169.9 mmol) and KOH (28.6 g, 509.7 mmol) were dissolved in EtOH (200 mL). The mixture was stirred at 80° C. for 24 hours. Evaporation of the solvent afforded a residue which was diluted in water, then the solution was extracted by ether. The aqueous phase was acidified at 0° C. to pH=1 with concentrated hydrochloric acid, then the precipitate was collected by suction filtration, washed with water, CH₂Cl₂ and dried in vacuo to afford 28.4 g (50% yield) of analytically pure 1 (yellow solid) confirmed by LCMS. Analytical LCMS (Method 1): single peak (214, 254 nm), t_{R}=1.65 min, MS (ESI⁺) m/z 279.09 (M+H)⁺.

### Methyl 2-(4-methoxyphenyl)quinoline-4-carboxylate (2)

To a solution of 1 (15 g, 53.8 mmol) in MeOH (50 mL) was added concentrated sulfuric acid (13 mL). The mixture was stirred under refluxing conditions overnight, and then diluted with water, extracted with ethyl acetate. The organic layer was separated, washed with brine, and dried over anhydrous Na₂SO₄ and filtered. The filtrate was evaporated under reduced pressure to afford 11.1 g (70%) of analytically pure 2 (yellow solid) confirmed by LCMS. Analytical LCMS (Method 2): single peak (214, 254 nm), t_{R}=2.05 min, MS (ESI⁺) m/z 293.11 (M+H)⁺.

### (2-(4-Methoxyphenyl)quinolin-4-yl)(pyridin-2-yl)methanone (3)

To a solution of n-BuLi (2.5 M, 26.4 mL, 66.1 mmol) in Et₂O (80 mL) cooled to −40° C. under N₂ was added a solution of 2-bromopyridine (10.4 g, 66.1 mmol) in Et₂O (20 mL). The reaction mixture was maintained at −78° C. for 20 minutes, then a solution of 2 (6.5 g, 22.0 mmol) in THF was added at −70° C. under N₂. The reaction mixture was stirred at −70° C. for 1.5 hours and then quenched with water, extracted with ethyl acetate. The organic phase was washed with brine and dried over anhydrous Na₂SO₄ and filtered. The filtrate was evaporated in vacuo to give a residue. The residue was triturated with Et₂O to afford 7 g (91%) of analytically pure 3 (yellow solid) confirmed by LCMS. Analytical LCMS (Method 1): single peak (214, 254 nm), t_{R}=1.87 min, MS (ESI⁺) m/z 341.2 (M+H)⁺.

### (2-(4-Methoxyphenyl)quinolin-4-yl)(pyridin-2-yl)methanol (4)

To a solution of 3 (7.7 g, 22.6 mmol) in EtOH (110 mL) cooled to 0° C. was added NaBH₄ (2.12 g, 45.3 mmol). After the addition is complete, the mixture was stirred for 0.5 hour and then quenched with water. Then ethyl acetate was added to the solution, the organic layer was separated, washed with brine, and dried over anhydrous sodium sulfate, and filtered. The filtrate was evaporated in vacuo to afford 4.6 g (60% yield) of 4 (white solid) which was used for the next step without further purification. Analytical LCMS (Method 2): single peak (214, 254 nm), t_{R}=1.53 min, MS (ESI⁺) 343.2 (M+H)⁺.

### (2-(4-Methoxyphenyl)quinolin-4-yl)(piperidin-2-yl)methanol (NSC23925)

To a solution of 4 (3.72 g, 10.88 mmol) in MeOH (360 mL) was added concentrated HCl (9 mL) and PtO₂ (741 mg, 3.26 mmol). The mixture was then hydrogenated at 20° C. for about 2 hours. The catalyst was filtered and washed with MeOH (3×10 mL), and the filtrate was concentrated under reduced pressure. The residue obtained was a dark green solid, which was used for the next step reaction without further purification. Chiral preparatory HPLC was used to separate the four diastereomers directly from NSC23925, however, all attempts failed. Analytical LCMS (Method 2): single peak (214, 254 nm), t_{R}=1.28 min, MS (ESI⁺) 349.3 (M+H)⁺.

### Tert-butyl 2-(hydroxy(2-(4-methoxy phenyl)quinolin-4-yl)methyl)piperidine-1-carboxylate (6 and 7)

To a solution of NSC23925 (the residue above) and Et₃N (3.48 g, 34.48 mmol) in THF (50 mL) cooled at 0° C. was added a solution of (Boc)₂O (4.0 g, 11.49 mmol) in THF (15 mL) dropwise. The mixture was stirred at room temperature overnight, and then water (50 mL) was added. The mixture was extracted with ethyl acetate (3×100 mL), the combined organic layers were washed with brine, dried over anhydrous sodium sulfate and filtered. The filtrate was evaporated in vacuo, and the crude residue was flash chromatographed on silica gel and eluted with 1:10˜1:6 EA/PE to afford 1.0 g of 6 (Analytical LCMS (Method 1): single peak (214, 254 nm), t_{R}=1.62 min, MS (ESI⁺) 449.0 (M+H)⁺) and 1.3 g of 7 (Analytical LCMS (Method 1): single peak (214, 254 nm), t_{R}=1.68 min, MS (ESI⁺) 449.3 (M+H)⁺).

### (2-(4-Methoxyphenyl)quinolin-4-yl)(piperidin-2-yl)methanol hydrochloride (NSC23925a˜d)

After purification of 1.3 g of 7 by chiral preparatory HPLC, two Boc protected chiral compounds were separated. Then each compound was de-protected with a solution of HCl in EtOH to give the corresponding HCl salts of NSC23925c (0.6 g, peak-1) and NSC23925d (0.6 g, peak-2). Using similar methods, NSC23925a (0.37 g, peak-1) and NSC23925b (0.26 g, peak-2) were prepared from 1 g of 6.

### (2-(4-methoxyphenyl)quinolin-4-yl)(piperidin-2-yl)methanol hydrochloride (NSC23925a (0.37 g, Peak-1))

¹H NMR (300 MHz, DMSO-d₆) δ 1.35 (m, 2H), 1.72 (m, 4H), 2.81 (m, 1H), 3.24 (m, 1H), 3.49 (m, 1H), 3.89 (s, 3H), 4.5 (brs, 1H), 5.68 (s, 1H), 7.20 (d, J=8.7 Hz, 2H), 7.74 (m, 1H), 7.93 (m, 1H), 8.26-8.38 (m, 5H), 8.53 (m, 1H), 9.25 (m, 1H); Analytical LCMS (Method 3): single peak (214, 254 nm), t_{R}=1.77 min, MS (ESI⁺) m/z 349.0 (M+H)⁺; chiral HPLC purity: 99.9% ee (t_{R} (major)=6.52 min, t_{R} (minor)=8.69 min).

### (2-(4-methoxyphenyl)quinolin-4-yl)(piperidin-2-yl)methanol hydrochloride (NSC23925b (0.26 g, Peak-2))

¹H NMR (300 MHz, DMSO-d₆) δ 1.35 (m, 2H), 1.72 (m, 4H), 2.81 (m, 1H), 3.24 (m, 1H), 3.49 (m, 1H), 3.89 (s, 3H), 4.5 (brs, 1H), 5.68 (s, 1H), 7.20 (d, J=8.7 Hz, 2H), 7.74 (m, 1H), 7.93 (m, 1H), 8.26-8.38 (m, 5H), 8.53 (m, 1H), 9.25 (m, 1H); Analytical LCMS (Method 3): single peak (214, 254 nm), t_{R}=1.78 min, MS (ESI⁺) m/z 349.0 (M+H)⁺; [α]_{D}²⁰=+15.0, (c=0.16, MeOH); chiral HPLC purity: 99.9% ee (t_{R} (major)=8.70 min, t_{R}(minor)=6.53 min).

### (2-(4-methoxyphenyl)quinolin-4-yl)(piperidin-2-yl)methanol hydrochloride (NSC23925c (0.6 g, Peak-1))

¹H NMR (300 MHz, DMSO-d₆): δ1.29 (m, 2H), 1.68 (m, 4H), 2.97 (m, 1H), 3.25 (m, 1H), 3.45 (m, 1H), 3.90 (s, 3H), 4.6 (brs, 1H), 6.11 (s, 1H), 7.23 (d, J=8.7 Hz, 2H), 7.78 (m, 1H), 8.01 (m, 1H), 8.26 (m, 3H), 8.50 (m, 2H), 8.69 (t, J=8.7 Hz, 1H), 10.46 (m, 1H); Analytical LCMS (Method 3): single peak (214, 254 nm), t_{R}=1.83 min, MS (ESI⁺) m/z 349.0 (M+H)⁺; [α]_{D}²⁰=−15.8 (c=0.2, MeOH); chiral HPLC purity: 99.9% ee (t_{R} (major)=4.96 min, t_{R} (minor)=12.7 min).

### (2-(4-methoxyphenyl)quinolin-4-yl)(piperidin-2-yl)methanol hydrochloride (NSC23925d (0.6 g, Peak-2))

¹H NMR (300 MHz, DMSO-d₆) δ 1.29 (m, 2H), 1.68 (m, 4H), 2.97 (m, 1H), 3.25 (m, 1H), 3.45 (m, 1H), 3.90 (s, 3H), 4.6 (brs, 1H), 6.11 (s, 1H), 7.23 (d, J=8.7 Hz, 2H), 7.78 (m, 1H), 8.01 (m, 1H), 8.26 (m, 3H), 8.50 (m, 2H), 8.69 (t, J=8.7 Hz, 1H), 10.46 (m, 1H); Analytical LCMS (Method 3): single peak (214, 254 nm), t_{R}=1.83 min, MS (ESI⁺) m/z 349.0 (M+H)⁺; [α]_{D}²⁰=+15.6 (c=0.2, MeOH); chiral HPLC purity: 97.2% ee (t_{R} (major)=12.7 min, t_{R}(minor)=4.7 min).

### Example 2

### Cell Lines, Cell Culture, and Drugs

A panel of parental cancer cell line and their daughter resistant cell lines, displaying MDR phenotype were used in this study. Human ovarian cancer cell line SKOV-3, breast cancer cell line MCF-7 and colon cancer cell line SW480, the osteosarcoma cell line U-2OS, KHOS, uterine sarcoma cell line MESSA and its doxorubicin selected drug resistant cell line MESSA/Dx5, non-small cell lung cancer cell line H-69 and its doxorubicin selected drug resistant cell line H-69AR (overexpress MRP1) were obtained from the ATCC (Rockville, Md.). Dr. Efstathios Gonos (Institute of Biological Research & Biotechnology, Athens, Greece) provided the doxorubicin resistant U-2OS R2 (referred in the text below as U-2OS_{DR}), KHOS R2 cell lines. Dr. Erasmus Schneider (Wadsworth Center, Albany) provided the mitoxantrane resistant breast cancer MCF-7/MX (overexpress BCRP) cell line. All the cell lines were cultured in RPMI 1640 (Invitrogen, Carlsbad, Calif.) supplemented with 10% fetal bovine serum, 100-units/ml penicillin and 100 μg/ml streptomycin (Invitrogen). Cells were incubated at 37° C. in 5% CO₂-95% air atmosphere and passaged when near confluent monolayers were achieved using 2% trypsin-EDTA solution. Drug-resistant cell lines were periodically cultured in the respective drug to confirm their drug resistance characteristics. The fresh chemotherapy drugs were obtained from the pharmacy at the Massachusetts General Hospital and stored at −20° C.

### Example 3

### Cytotoxicity MTT Assay and Reversals of MDR

To evaluate for reversal of Pgp-mediated MDR, SKOV-3_{TR}, MCF-7adr and SW480_{TR} cell lines were seeded into 96-well tissue culture plate at 2,000 cells per well with varying concentrations of chemotherapy drugs and NSC23925b for 6 days. Cell proliferation IC₅₀ (concentration required for 50% inhibition) and MDR reversal IC₅₀ were determined from dose-response curves carried out in triplicate in 96-well plate. Fold of reverse resistance for individual drugs were defined as the IC₅₀ of MDR cells without NSC23925b treatment divided by the IC₅₀ of MDR cells treated with NSC23925b. To test for the effect of reversal of MRP1 or BCRP-mediated MDR, H-69AR or MCF-7/MX cells were placed into 96-wells and treated with chemotherapy drugs similar as described above. Drug cytotoxicity was assessed in vitro using the MTT assay as previously described. Drugs at the concentrations utilized in the MTT assay were performed in the absence of cells to verify no change in absorbance. Response curves were fitted with use of GraphPad PRISM 4 software (GraphPad Software).

### Example 4

### Drug Efflux Fluorescence Microscopy Assay

The Vybrant™ multi-drug resistance assay kit (Invitrogen/Molecular Probes) was used to measure drug efflux properties of different resistant cell lines. This assay utilizes the fluorogenic dye calcein acetoxymethyl ester (calcein AM) as a substrate for efflux activity of Pgp or other membrane pump ABC proteins. Calcein AM is taken up by cells and hydrolyzed by cytoplasmic esterases to fluorescent calcein. Calcein AM is well retained in the cytosol. However, multidrug resistant cells expressing high levels of Pgp rapidly extrude non-fluorescent calcein AM from the plasma membrane, reducing accumulation of fluorescent calcein in the cytosol. Studies were carried out as described. In brief, drug resistant cells (5,000 cells per well) were cultured in 96-well plate for 24 hours. Triplicated cultures of cells were treated with different concentrations of NSC23925b for one hour and then incubated in calcein AM in 100 μl total volume. After 30 minutes, images were acquired by Nikon Eclipse Ti-U fluorescence microscope (Nikon Corp.) equipped with a SPOT RT digital camera (Diagnostic Instruments, Inc., Sterling Heights, Mich.).

### Example 5

### Apoptosis Assays

Apoptosis was evaluated using the M30-Apoptosense ELISA assay kit (Peviva AB, Bromma, Sweden) according to the manufacturer's instructions. For the doxorubicin and NSC23925b treatment, MCF-7adr or SW480_{TR} cells were seeded at 8,000 cells/per well in a 96-well plate for 24 hours before treatment. The cells were then treated with 0.5 μM of doxorubicin, 1 μM of NSC23925b, or a combination of the two drugs for an additional 72 hours. The cells were then lysed by an additional 10 AL of 10% NP40 per well, following the manufacturer's instructions for apoptosis assay.

### Example 6

### Effect of Combination of NSC23925b and Paclitaxel on Tumor Growth In Vivo

The Crl:SHO-Prkdc^{SCID}Hr^{hr} nude female mice at approximately 3-4 weeks of age were purchased from The Charles River Laboratories (Ann Arbor, Mich.). Animal studies were carried out with protocols approved by the Massachusetts General Hospital Subcommittee on Research Animal Care (SRAC). To determine the effect of NSC23925b on paclitaxel sensitivity in xenograft model, 5×10⁶ SKOV-3_{TR} cells were injected subcutaneously with matrigel (BD Biosciences, San Jose, Calif.) on day one. Two weeks after injection, the mice were divided into four groups to start the treatments. Group one was received intraperitoneal (i.p) injection with normal saline, group two with paclitaxel (20 mg/kg), group three with NSC23925b (40 mg/kg). The final group of mice received paclitaxel (20 mg/kg) and NSC23925b (40 mg/kg). All four groups were treated twice a week for two weeks. The health of the mice and evidence of tumor growth were examined daily. Tumors were measured twice a week with a digital caliper. Tumor volume (mm³) was calculated as (W²×L)/2, where W is width and L is length. The curve of tumor growth was drawn according to tumor volume by using of GraphPad PRISM 4 software.

### Example 7

### Identification of NSC23925b as a Most Potent Compound that Reverses MDR in Human Cancer Cell Lines

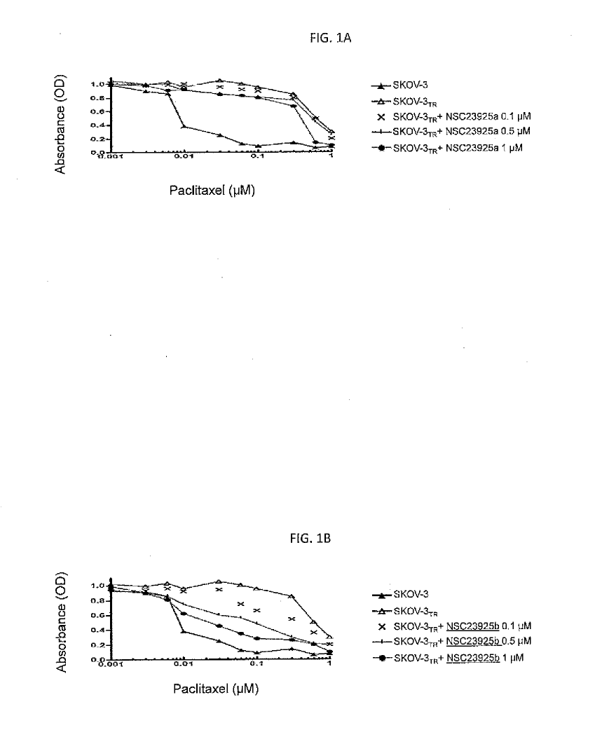

Structure and activity relationship (SAR) of the four NSC23925 isomers for reversing MDR were evaluated in human ovarian cancer MDR cell line SKOV-3_{TR} by MTT assay (FIGS. 1A-D). Cells were treated with the paclitaxel and NSC23925 diastereomers NSC23925a (FIG. 1A), NSC23925b (FIG. 1B), NSC23925c (FIG. 1C) and NSC23925d (FIG. 1D) in RPMI1640 complete media at the indicated concentrations. The relative sensitivity of each line to paclitaxel was determined by MTT analysis 6 days post-treatment. NSC23925b was determined to be the most potent isomer on reversing drug resistance, NSC23925c and NSC23925d also showed modest but lesser activity, and NSC23925a showed the least potency.

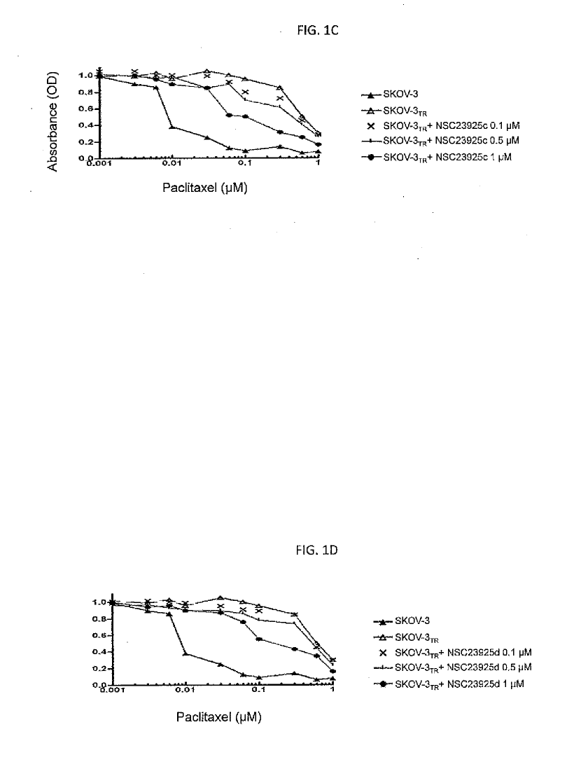

Further studies showed NSC23925b was able to reverse paclitaxel, doxorubicin, and mitoxantrane resistance in human breast cancer MDR cell line MCF-7adr and human colon cancer MDR cell line SW480_{TR} (FIGS. 2A-F). MCF-7adr and SW480TR cells were treated with paclitaxel (FIGS. 2A-B), doxorubicin (FIGS. 2C-D) or mitoxantrane (FIGS. 2E-F) and NSC23925b in RPMI1640 complete media at the indicated concentrations. The relative sensitivity of each line to the indicated drugs was determined by MTT analysis 6 days post-treatment.

NSC23925b was able to reverse MDR in several MDR cell lines, including ovarian, breast, uterine, colon, and sarcoma MDR cancer cell lines, to paclitaxel, doxorubicin, and mitoxantrane (Table 1 below). The results also showed NSC23925b had no significant effects on the drug sensitivities of cisplatin, carboplatin, toptecan and methotrexate (Table 2 below). Furthermore, the effects of NSC23925b on MDR1 (Pgp) independent MRP1 or BCRP mediated drug resistance in H-69AR, MCF-7/MX cell lines were evaluated. The results showed NSC23925b was unable to reverse drug resistance in these non-Pgp expressing drug resistant cell lines, suggesting that NSC23925b activity is specific for Pgp.

TABLE 1
Effect of NSC23925b on Reverse Drug Resistance in MDR Cell Lines

| | SKOV-3_{TR} | MCF-7adr | SW480_{TR} |
|---|---|---|---|
| | IC₅₀ (μM) | IC₅₀ (μM) | IC₅₀(μM) |
| Paclitaxel | 0.57 ± 0.03 | 0.62 ± 0.12 | 0.12 ± 0.08 |
| With NSC23925b | 0.33 ± 0.04 (2) | 0.46 ± 0.1 (1.3) | 0.09 ± 0.01 (1.3) |
| 0.1 μM | | | |
| With NSC23925b | 0.08 ± 0.06 (7) | 0.16 ± 0.03 (4) | 0.016 ± 0.006 (8) |
| 0.5 μM | | | |
| With NSC23925b | 0.02 ± 0.03 (28) | 0.05 ± 0.02 (12) | 0.009 ± 0.002 (13) |
| 1 μM | | | |
| Doxorubicin | 38.5 ± 2.6 | 3.8 ± 0.12 | 0.84 ± 0.16 |
| With NSC23925b | 24.6 ± 1.2 (2) | 2.8 ± 0.4 (1.4) | 0.5 ± 0.1 (1.7) |
| 0.1 μM | | | |
| With NSC23925b | 4.2 ± 0.5 (9) | 0.96 ± 0.22 (4) | 0.28 ± 0.08 (3) |
| 0.5 μM | | | |
| With NSC23925b | 1.2 ± 0.3 (32) | 0.25 ± 0.06 (15) | 0.11 ± 0.04 (8) |
| 1 μM | | | |
| Mitoxantrane | 0.46 ± 0.06 | 0.32 ± 0.06 | 0.27 ± 0.03 |
| With NSC23925b | 0.37 ± 0.05 (1.2) | 0.3 ± 0.001 (1.1) | 0.23 ± 0.03 (1.2) |
| 0.1 μM | | | |
| With NSC23925b | 0.18 ± 0.02 (2.6) | 0.12 ± 0.02 (3) | 0.09 ± 0.03 (3) |
| 0.5 μM | | | |
| With NSC23925b | 0.06 ± 0.01 (8) | 0.05 ± 0.01 (6) | 0.02 ± 0.01 (14) |
| 1 μM | | | |

Cell survival was determined by MTT assay as described in Example 1.
IC₅₀ is the concentration of drug (μM) that produced 50% inhibition of cell growth.
Results were calculated from one experiment with triplicate wells.
The number in the parentheses represents fold-reversal of drug resistance.

TABLE 2
Reverse Drug Resistance by NSC23925b

| Decreased Drug Resistance | No Effect |
|---|---|
| Paclitaxel | Cisplatin |
| Docetaxel | Carboplatin |
| Doxorubicin | Topotecan |
| Vincristine | Methotrexate |
| Daunorubicin | |
| Gemcitabine | |
| ET-743 | |
| Mitoxantrane | |

### Example 8

### NSC23925b Modulates Pgp-Mediated Uptake and Efflux of Calcein AM

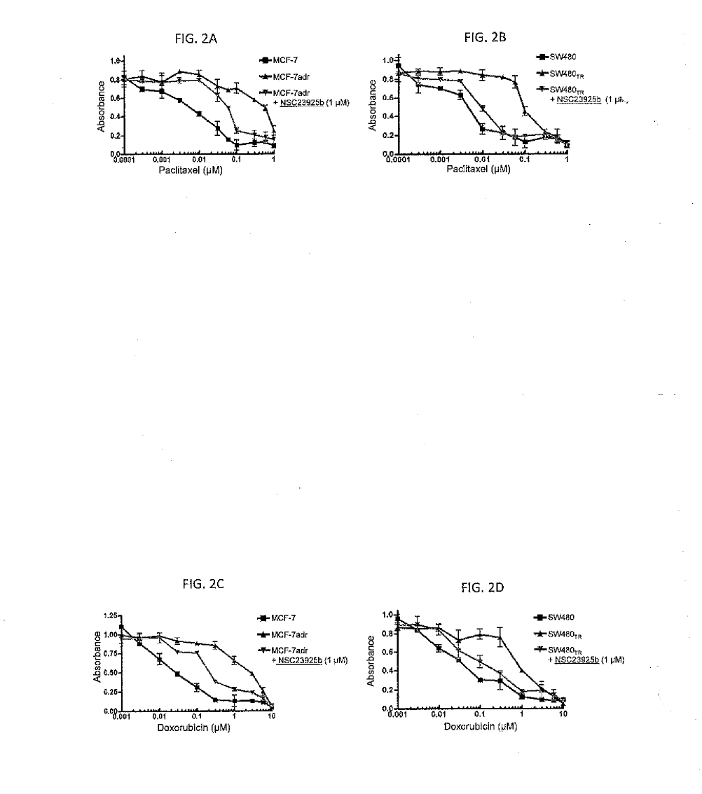

Reverse of MDR is usually displayed as an increased intracellular accumulation of chemotherapeutic drugs. The effect of NSC23925b on the uptake and efflux of calcein AM in MCF-7adr and SW480_{TR} was determined. Fluorescence microscopic analysis showed NSC23925b was able to increase intracellular accumulation of calcein AM in these cell lines in a dose-dependent manner (FIG. 3). The calcein-AM assay was optimized and performed using the Vybrant Multidrug Resistance kit. Cells were seeded at 50,000 cells/well (100 μl of culture medium) in 96-well plate and incubated for 24 hours. MCF-7adr cells in triplicate were treated with NSC23925b for one hour and then incubated in calcein AM for 30 minutes. The cell fluorescence images were acquired by a fluorescence microscope. NSC23925b has a prominent effect on the accumulation of calcein AM in these cells starting at a concentration as low as 1 nM. Half-maximal reversal of accumulation deficit was observed at 100 nM and near maximal at 500 nM.

### Example 9

### Combination of NSC23925b and Doxorubincin Induces Apoptosis in MDR Cancer Cells

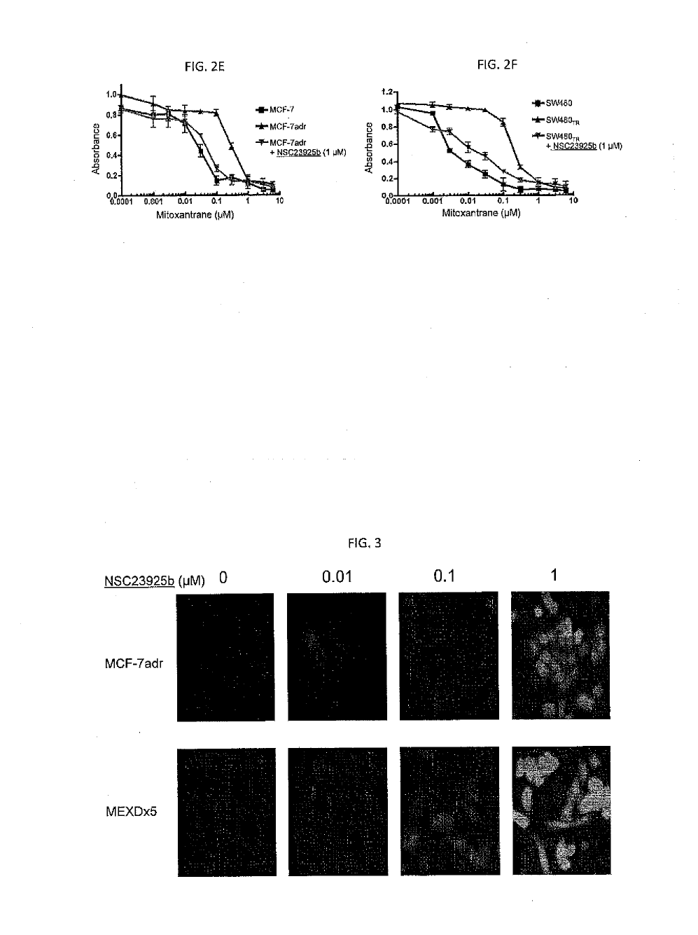

The effect of NSC23925b on the doxorubicin induction of apoptosis was assessed in MCF-7adr or SW480_{TR}. cell lines. Cells were treated with either doxorubicin (0.5 μM) alone, NSC23925b (1 μM) alone, or a combination of doxorubicin and NSC23925b for 72 hours. Apoptosis was scored using the M30-Apoptosense ELISA assay. The combination of doxorubicin and NSC23925b resulted in significantly greater cell death as compared with doxorubicin or NSC23925b alone (FIGS. 4A and 4B). Additionally, 3-(4,5-dimethylthiazol-2-yl)-2,5-diphenyltetrazolium bromide cytotoxicity assay also showed that NSC23925b had a synergistic effect on doxorubicin-induced cell death.

### Example 10

### Effect of Combination Therapy with NSC23925b on Tumor Growth In Vivo

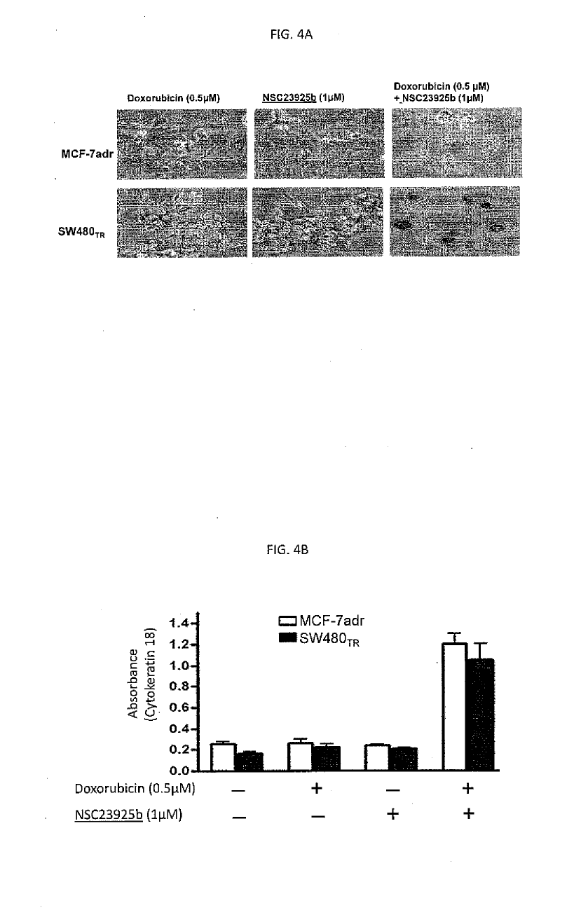

To further evaluate the effects of NSC23925b and paclitaxel combination therapy on the tumor growth of resistant cells, the growth of xenograft tumors were examined. A well-characterized ovarian cancer MDR cell line SKOV-3_{TR} was chosen for in vivo studies. The results showed that NSC23925b or paclitaxel alone had no significant effects on the paclitaxel resistant tumor cell growth by themselves. However, the combination of paclitaxel and NSC23925b produced a significantly greater inhibitory effect on tumor growth on paclitaxel resistant tumors when compared with animals treated with NSC23925b, paclitaxel (FIGS. 5A and 5B). Tumors were measured twice a week with a digital caliper. Tumor volume (mm³) was calculated as (W²×L)/2, where W is width and L is length (FIG. 5A). Representative images of dissected tumor tissues from treated nude mice. NSC23925b (50 mg/kg) were found to almost completely reverse resistance to paclitaxel in this model (FIG. 5B). In addition, on the basis of animal weight and mortality, no significant toxicity was observed and the animals appeared to be well tolerated when NSC23925b was administered alone. Instead, when combined with paclitaxel at the concentrations indicated, NSC23925b showed reducing paclitaxel-induced toxicity in some animals.

### Example 11

### Structural Elucidation of NSC23925b

Chiral HPLC Analysis.

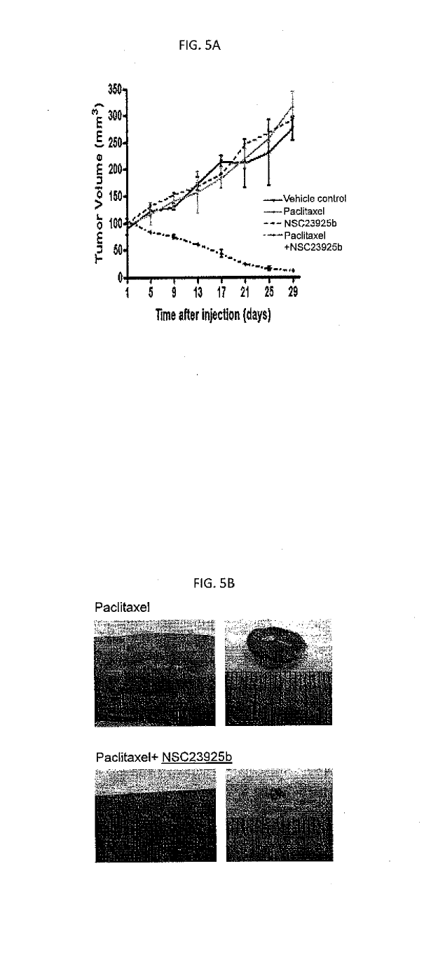

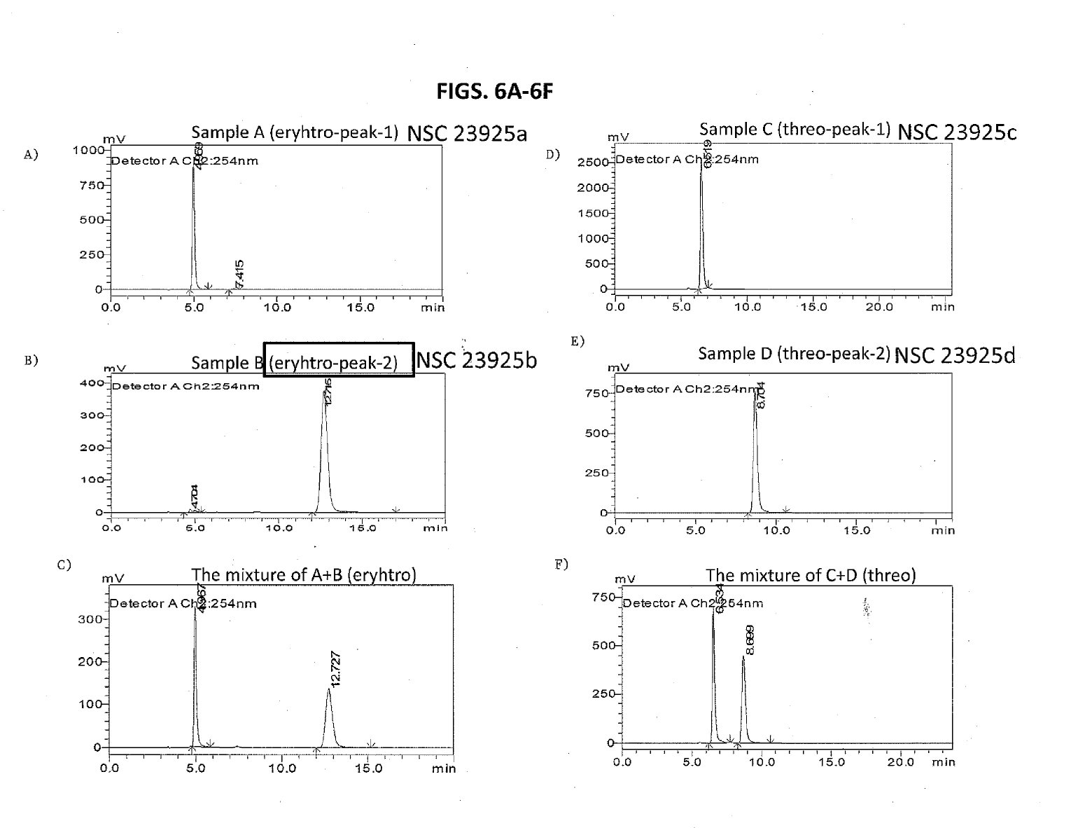

FIGS. 6A-6F include chiral HPLC traces for each of NSC23925a, NSC23925b, NSC23925c, and NSC23925d and erythro and threo mixtures thereof. FIG. 7 is an LCMS trace showing the chemical purity of NSC 23925 from the Structural Diversity Set library.

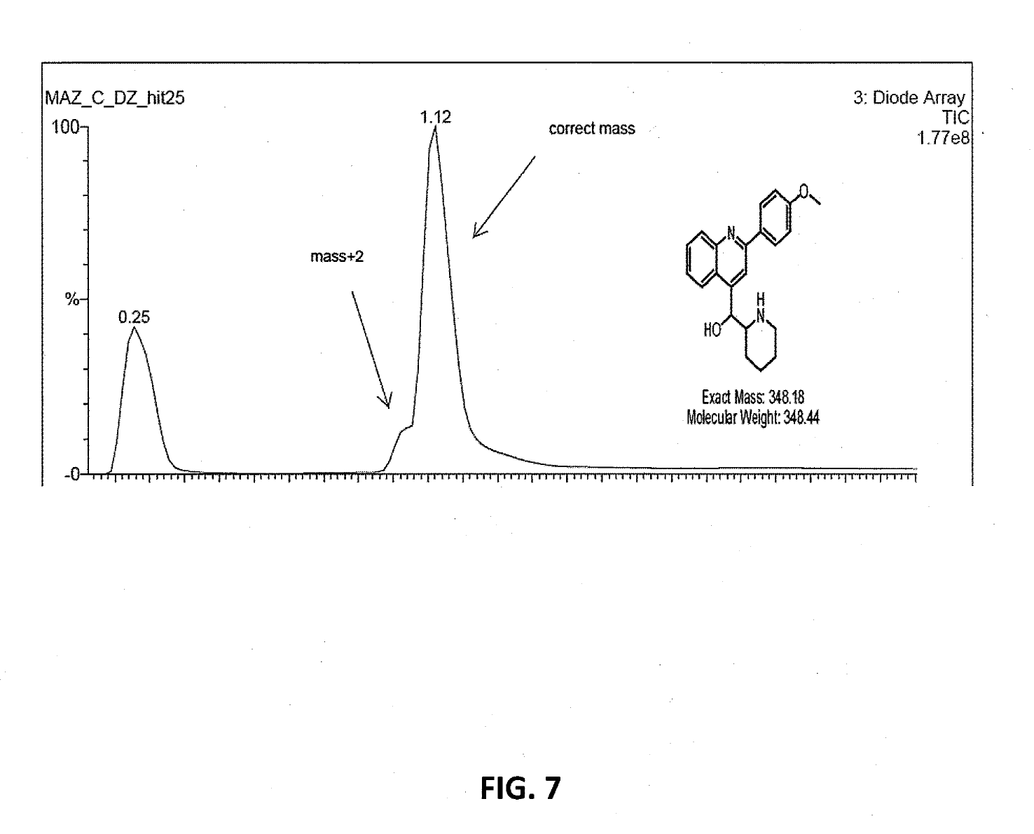

FIG. 8 is a chiral HPLC trace of NSC 23925 from the Structural Diversity Set library. This trace was obtained using a chiral HPLC method that was developed for the analysis and separation of synthetically prepared NSC23925a, NSC23925b, NSC23925c, and NSC23925d. As can be seen, NSC 23925 from the Structural Diversity Set library includes some amount of each of the four possible (2-(4-methoxy)quinolin-4-yl)(piperidin-2-yl)methanol stereoisomers.

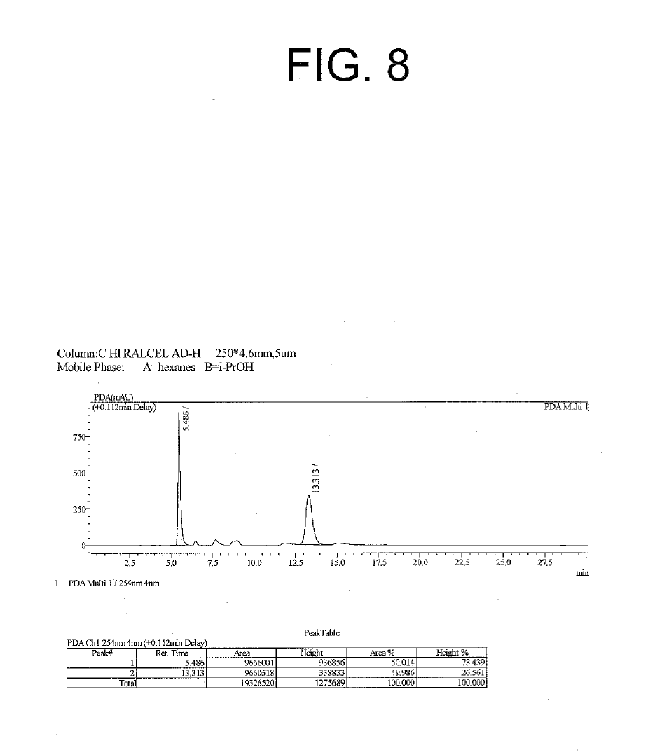

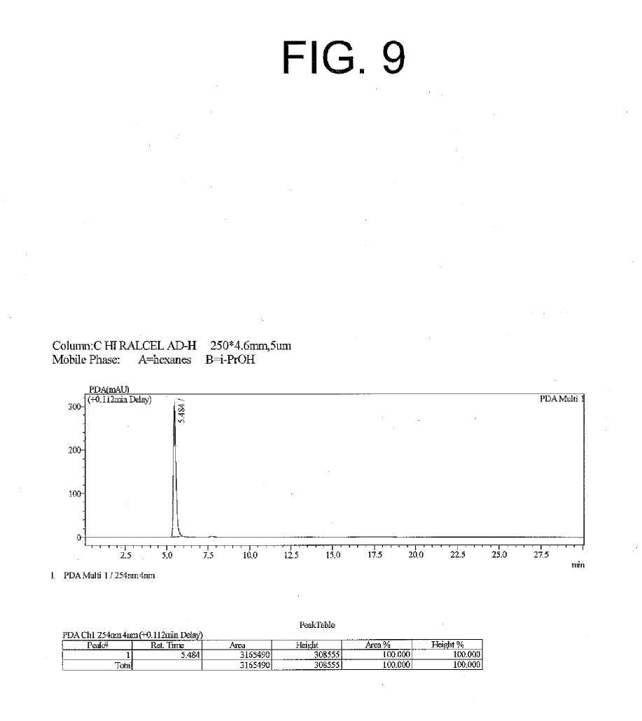

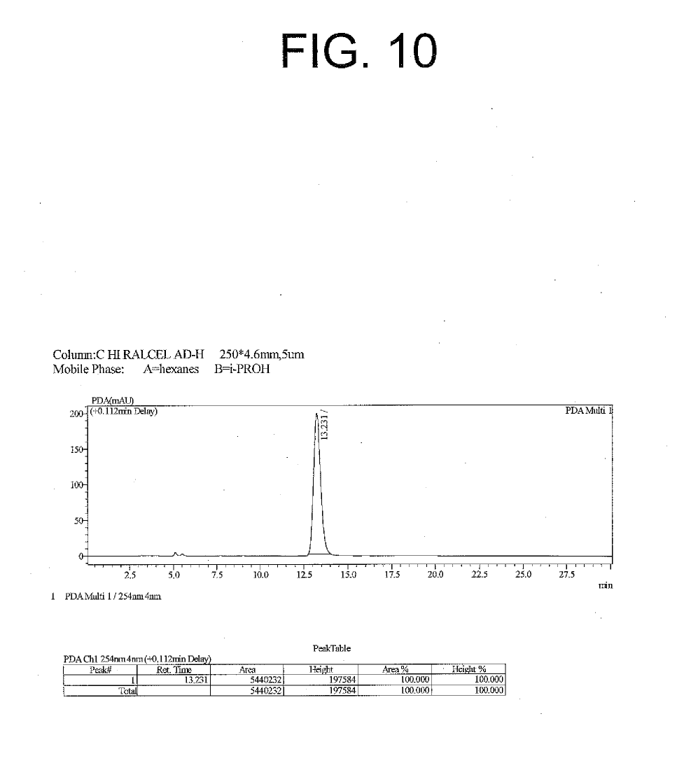

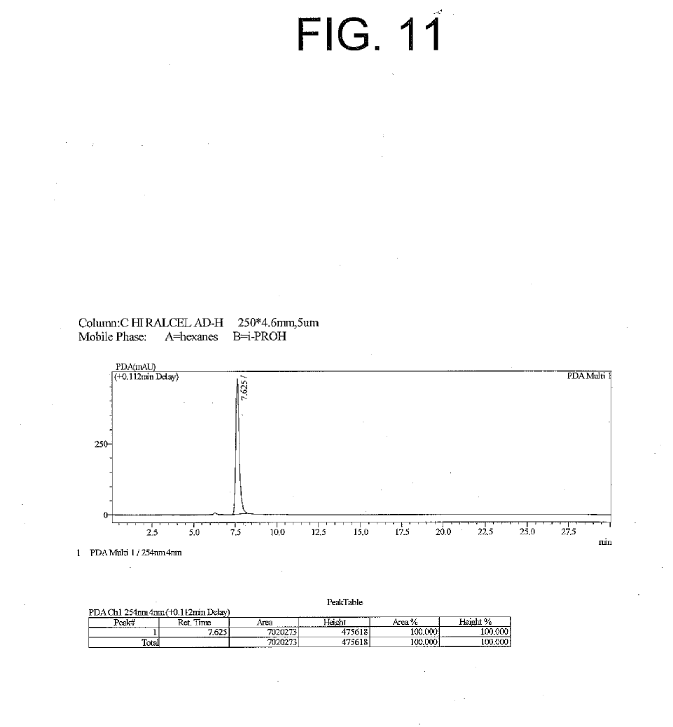

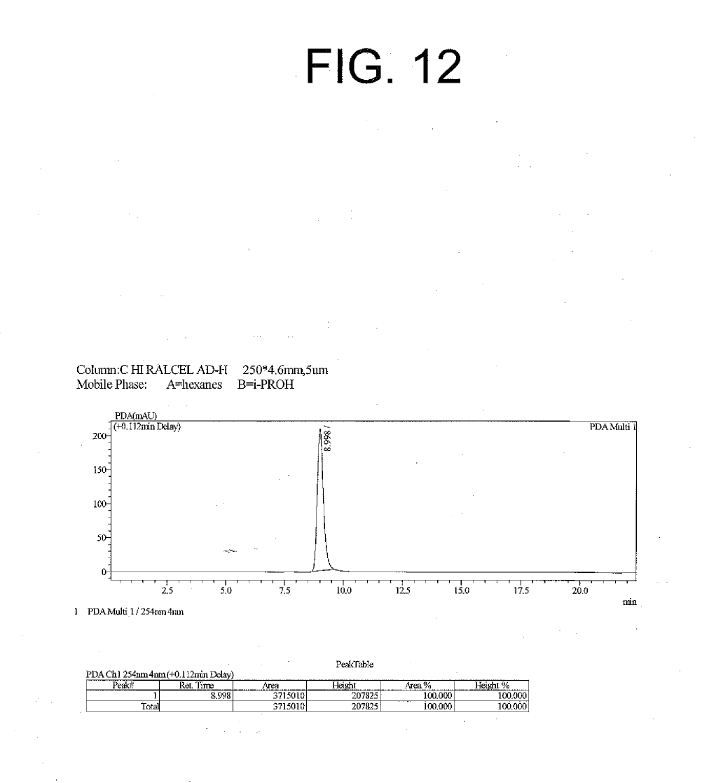

FIG. 9 is a chiral HPLC trace of a purified sample of synthetically prepared NSC 23925a. This trace was obtained using the same set of conditions used to obtain the trace shown in FIG. 8. FIG. 10 is a chiral HPLC trace of a purified sample of synthetically prepared NSC 23925b. This trace was obtained using the same set of conditions used to obtain the trace shown in FIG. 8. FIG. 11 is a chiral HPLC trace of a purified sample of synthetically prepared NSC 23925c. This trace was obtained using the same set of conditions used to obtain the trace shown in FIG. 8. FIG. 12 is a chiral HPLC trace of a purified sample of synthetically prepared NSC 23925d. This trace was obtained using the same set of conditions used to obtain the trace shown in FIG. 8. FIG. 13 is a chiral HPLC trace of a mixture containing purified, synthetically prepared NSC 23925a, purified, synthetically prepared NSC 23925b, purified, synthetically prepared NSC 23925c, and purified, synthetically prepared NSC 23925d. This trace was obtained using the same set of conditions used to obtain the trace shown in FIG. 8.

X-Ray Crystallography

Using the chiral HPLC methods described above and in the drawings, a sample of NSC 23925b was isolated and crystallized as described below.

X-Ray Crystallography:

A crystal mounted on a diffractometer was collected data at 100 K. The intensities of the reflections were collected by means of a Bruker APEX II CCD diffractometer (Mo_{Kα} radiation, λ=0.71073 Å), and equipped with an Oxford Cryosystems nitrogen flow apparatus. The collection method involved 0.5° scans in ω at 28° in 2θ Data integration down to 0.76 Å resolution was carried out using SAINT V7.46 A (Bruker diffractometer, 2009) with reflection spot size optimization. Absorption corrections were made with the program SADABS (Bruker diffractometer, 2009). The structure was solved by the direct methods procedure and refined by least-squares methods again F² using SHELXS-97 and SHELXL-97 (Sheldrick, 2008). Non-hydrogen atoms were refined anisotropically, and hydrogen atoms were allowed to ride on the respective atoms. Crystal data as well as details of data collection and refinement are summarized in Table 3, geometric parameters are shown in Table 4, and hydrogen-bond parameters are listed in Table 5. The Ortep plots produced with SHELXL-97 program, and the other drawings were produced with Accelrys DS Visualizer 2.0 (Accelrys, 2007).

TABLE 3
Crystal data

| Chemical formula | C₂₂H₂₆Cl₂N₂O₂ |
|---|---|
| Mᵣ | 421.35 |
| Crystal system, space group | Orthorhombic, P2₁2₁2₁ |
| Temperature (K) | 100 |
| a, b, c (Å) | 6.8953 (2), 9.8443 (3), 29.9185 (9) |
| V (Å³) | 2030.85 (11) |
| Z | 4 |
| Radiation type | Mo Kα |
| μ (mm⁻¹) | 0.34 |
| Crystal size (mm) | 0.24 × 0.18 × 0.16 |

Data collection

| | |
|---|---|
| Diffractometer | Bruker D8 goniometer with CCD |
| | area detector diffractometer |
| Absorption correction | Multi-scan SADABS |
| Tₘᵢₙ, Tₘₐₓ | 0.923, 0.948 |
| No. of measured, independent | 26991, 4850, 4241 |
| and observed [I > 2σ(I)] | |
| reflections | |
| Rᵢₙₜ | 0.066 |

Refinement

| | |
|---|---|
| R[F² > 2σ(F²)], wR(F²), S | 0.038, 0.094, 1.03 |
| No. of reflections | 4850 |
| No. of parameters | 270 |
| No. of restraints | 0 |
| H-atom treatment | H atoms treated by a mixture of |
| | independent and constrained refinement |
| Δρₘₐₓ, Δρₘᵢₙ (e Å⁻³) | 0.43, −0.27 |
| Absolute structure | Flack H D (1983), |
| | Acta Cryst. A39, 876-881 |
| Flack parameter | −0.05 (6) |

Computer programs: APEX2 v2009.3.0 (Bruker-AXS, 2009), SAINT 7.46A (Bruker-AXS, 2009), SHELXS97 (Sheldrick, 2008), SHELXL97 (Sheldrick, 2008), Bruker SHELXTL (Sheldrick, 2008).

TABLE 4
Geometric parameters (Å, °)

| O1—C13 | 1.354 | (2) | C10—C11 | 1.408 | (3) |
|---|---|---|---|---|---|
| O1—C16 | 1.438 | (3) | C11—C12 | 1.377 | (3) |
| O2—C17 | 1.411 | (3) | C11—H11 | 0.9500 | |
| O2—H2 | 0.83 | (3) | C12—C13 | 1.394 | (3) |
| N1—C1 | 1.339 | (3) | C12—H12 | 0.9500 | |
| N1—C9 | 1.386 | (3) | C13—C14 | 1.396 | (3) |
| N1—H1 | 0.93 | (3) | C14—C15 | 1.387 | (3) |
| N2—C18 | 1.497 | (3) | C14—H14 | 0.9500 | |
| N2—C22 | 1.501 | (3) | C15—H15 | 0.9500 | |
| N2—H2A | 0.89 | (3) | C16—H16A | 0.9800 | |
| N2—H2B | 0.91 | (3) | C16—H16B | 0.9800 | |
| C1—C2 | 1.414 | (3) | C16—H16C | 0.9800 | |
| C1—C10 | 1.473 | (3) | C17—C18 | 1.538 | (3) |
| C2—C3 | 1.367 | (3) | C17—H17 | 1.0000 | |
| C2—H2C | 0.9500 | | C18—C19 | 1.517 | (3) |
| C3—C4 | 1.430 | (3) | C18—H18 | 1.0000 | |
| C3—C17 | 1.524 | (3) | C19—C20 | 1.529 | (3) |
| C4—C5 | 1.418 | (3) | C19—H19A | 0.9900 | |
| C4—C9 | 1.418 | (3) | C19—H19B | 0.9900 | |
| C5—C6 | 1.370 | (3) | C20—C21 | 1.527 | (3) |
| C5—H5 | 0.9500 | | C20—H20A | 0.9900 | |
| C6—C7 | 1.404 | (3) | C20—H20B | 0.9900 | |
| C6—H6 | 0.9500 | | C21—C22 | 1.513 | (3) |
| C7—C8 | 1.377 | (3) | C21—H21A | 0.9900 | |
| C7—H7 | 0.9500 | | C21—H21B | 0.9900 | |
| C8—C9 | 1.400 | (3) | C22—H22A | 0.9900 | |
| C8—H8 | 0.9500 | | C22—H22B | 0.9900 | |
| C10—C15 | 1.396 | (3) | | | |
| C13—O1—C16 | 117.37 | (16) | C12—C13—C14 | 120.01 | (19) |
| C17—O2—H2 | 113 | (2) | C15—C14—C13 | 119.0 | (2) |
| C1—N1—C9 | 123.26 | (18) | C15—C14—H14 | 120.5 | |
| C1—N1—H1 | 125.0 | (19) | C13—C14—H14 | 120.5 | |
| C9—N1—H1 | 111.5 | (19) | C14—C15—C10 | 121.71 | (19) |
| C18—N2—C22 | 112.96 | (17) | C14—C15—H15 | 119.1 | |
| C18—N2—H2A | 111.6 | (16) | C10—C15—H15 | 119.1 | |
| C22—N2—H2A | 110.9 | (16) | O1—C16—H16A | 109.5 | |
| C18—N2—H2B | 105.3 | (16) | O1—C16—H16B | 109.5 | |
| C22—N2—H2B | 108.3 | (16) | H16A—C16—H16B | 109.5 | |
| H2A—N2—H2B | 108 | (2) | O1—C16—H16C | 109.5 | |
| N1—C1—C2 | 117.84 | (19) | H16A—C16—H16C | 109.5 | |
| N1—C1—C10 | 121.26 | (18) | H16B—C16—H16C | 109.5 | |
| C2—C1—C10 | 120.84 | (19) | O2—C17—C3 | 113.08 | (17) |
| C3—C2—C1 | 122.2 | (2) | O2—C17—C18 | 105.72 | (17) |
| C3—C2—H2C | 118.9 | | C3—C17—C18 | 110.82 | (17) |
| C1—C2—H2C | 118.9 | | O2—C17—H17 | 109.0 | |
| C2—C3—C4 | 119.3 | (2) | C3—C17—H17 | 109.0 | |
| C2—C3—C17 | 119.2 | (2) | C18—C17—H17 | 109.0 | |
| C4—C3—C17 | 121.55 | (19) | N2—C18—C19 | 109.85 | (17) |
| C5—C4—C9 | 117.55 | (19) | N2—C18—C17 | 107.80 | (16) |
| C5—C4—C3 | 124.6 | (2) | C19—C18—C17 | 114.20 | (17) |
| C9—C4—C3 | 117.82 | (19) | N2—C18—H18 | 108.3 | |
| C6—C5—C4 | 120.8 | (2) | C19—C18—H18 | 108.3 | |
| C6—C5—H5 | 119.6 | | C17—C18—H18 | 108.3 | |
| C4—C5—H5 | 119.6 | | C18—C19—C20 | 110.87 | (17) |
| C5—C6—C7 | 120.3 | (2) | C18—C19—H19A | 109.5 | |
| C5—C6—H6 | 119.9 | | C20—C19—H19A | 109.5 | |
| C7—C6—H6 | 119.9 | | C18—C19—H19B | 109.5 | |
| C8—C7—C6 | 121.0 | (2) | C20—C19—H19B | 109.5 | |
| C8—C7—H7 | 119.5 | | H19A—C19—H19B | 108.1 | |
| C6—C7—H7 | 119.5 | | C21—C20—C19 | 109.86 | (17) |
| C7—C8—C9 | 118.89 | (19) | C21—C20—H20A | 109.7 | |
| C7—C8—H8 | 120.6 | | C19—C20—H20A | 109.7 | |
| C9—C8—H8 | 120.6 | | C21—C20—H20B | 109.7 | |
| N1—C9—C8 | 119.07 | (19) | C19—C20—H20B | 109.7 | |
| N1—C9—C4 | 119.49 | (18) | H20A—C20—H20B | 108.2 | |
| C8—C9—C4 | 121.42 | (19) | C22—C21—C20 | 110.68 | (18) |
| C15—C10—C11 | 118.34 | (18) | C22—C21—H21A | 109.5 | |
| C15—C10—C1 | 119.91 | (18) | C20—C21—H21A | 109.5 | |
| C11—C10—C1 | 121.69 | (18) | C22—C21—H21B | 109.5 | |
| C12—C11—C10 | 120.2 | (2) | C20—C21—H21B | 109.5 | |
| C12—C11—H11 | 119.9 | | H21A—C21—H21B | 108.1 | |
| C10—C11—H11 | 119.9 | | N2—C22—C21 | 110.73 | (18) |
| C11—C12—C13 | 120.69 | (19) | N2—C22—H22A | 109.5 | |
| C11—C12—H12 | 119.7 | | C21—C22—H22A | 109.5 | |
| C13—C12—H12 | 119.7 | | N2—C22—H22B | 109.5 | |
| O1—C13—C12 | 115.94 | (18) | C21—C22—H22B | 109.5 | |
| O1—C13—C14 | 124.05 | (19) | H22A—C22—H22B | 108.1 | |
| C9—N1—C1—C2 | −0.6 | (3) | C1—C10—C11—C12 | 176.08 | (19) |
| C9—N1—C1—C10 | −177.74 | (18) | C10—C11—C12—C13 | −0.1 | (3) |
| N1—C1—C2—C3 | −2.5 | (3) | C16—O1—C13—C12 | 178.35 | (19) |
| C10—C1—C2—C3 | 174.69 | (18) | C16—O1—C13—C14 | −1.3 | (3) |
| C1—C2—C3—C4 | 2.9 | (3) | C11—C12—C13—O1 | −178.80 | (18) |
| C1—C2—C3—C17 | −175.39 | (19) | C11—C12—C13—C14 | 0.8 | (3) |
| C2—C3—C4—C5 | −179.57 | (19) | O1—C13—C14—C15 | 179.2 | (2) |
| C17—C3—C4—C5 | −1.3 | (3) | C12—C13—C14—C15 | −0.4 | (3) |
| C2—C3—C4 —C9 | −0.4 | (3) | C13—C14—C15—C10 | −0.8 | (3) |
| C17—C3—C4—C9 | 177.87 | (18) | C11—C10—C15—C14 | 1.6 | (3) |
| C9—C4—C5—C6 | 2.5 | (3) | C1—C10—C15—C14 | −175.67 | (19) |
| C3—C4—C5—C6 | −178.3 | (2) | C2—C3—C17—O2 | 10.2 | (3) |
| C4—C5—C6—C7 | −1.7 | (3) | C4—C3—C17—O2 | −168.15 | (18) |
| C5—C6—C7—C8 | −0.2 | (3) | C2—C3—C17—C18 | −108.3 | (2) |
| C6—C7—C8—C9 | 1.2 | (3) | C4—C3—C17—C18 | 73.4 | (2) |
| C1—N1—C9—C8 | −178.6 | (2) | C22—N2—C18—C19 | −56.2 | (2) |
| C1—N1—C9—C4 | 3.0 | (3) | C22—N2—C18—C17 | 178.77 | (18) |
| C7—C8—C9—N1 | −178.6 | (2) | O2—C17—C18—N2 | 66.3 | (2) |
| C7—C8—C9—C4 | −0.3 | (3) | C3—C17—C18—N2 | −170.88 | (17) |
| C5—C4—C9—N1 | 176.76 | (18) | O2—C17—C18—C19 | −56.1 | (2) |
| C3—C4—C9—N1 | −2.4 | (3) | C3—C17—C18—C19 | 66.8 | (2) |
| C5—C4—C9—C8 | −1.6 | (3) | N2—C18—C19—C20 | 56.7 | (2) |
| C3—C4—C9—C8 | 179.2 | (2) | C17—C18—C19—C20 | 177.96 | (18) |
| N1—C1—C10—C15 | −162.2 | (2) | C18—C19—C20—C21 | −57.6 | (2) |
| C2—C1—C10—C15 | 20.7 | (3) | C19—C20—C21—C22 | 56.8 | (2) |
| N1—C1—C10—C11 | 20.6 | (3) | C18—N2—C22—C21 | 56.0 | (2) |
| C2—C1—C10—C11 | −156.5 | (2) | C20—C21—C22—N2 | −55.6 | (2) |
| C15—C10—C11—C12 | −1.1 | (3) | | | |

TABLE 5
Hydrogen-bond parameters

| | D-H | H . . . A | D . . . A | D-H . . . A |
|---|---|---|---|---|
| D-H . . . A | (Å) | (Å) | (Å) | (°) |
| O2—H2 . . . Cl1ⁱ | 0.83 (3) | 2.29 (3) | 3.1046 (17) | 168 (3) |
| N1—H1 . . . Cl1 | 0.93 (3) | 2.19 (3) | 3.1151 (18) | 176 (3) |
| N2—H2A . . . Cl2 | 0.89 (3) | 2.35 (3) | 3.123 (2) | 145 (2) |
| N2—H2B . . . Cl2ⁱⁱ | 0.91 (3) | 2.16 (3) | 3.0640 (19) | 171 (2) |
| N2—H2A . . . O2 | 0.89 (3) | 2.49 (2) | 2.824 (2) | 102.5 (18) |

Symmetry code (s):
ⁱx + 1/2, −y + 1/2, −z + 1;
ⁱⁱ−x + 1, y − 1/2, −-z + 1/2.

FIG. 14 shows the chemical structure of NSC 23925b with absolute stereochemistry as determined by x-ray crystallography. The image includes perspective views showing 50% probability displacement ellipsoids. As can be seen, C-17 in NSC 23925b has the S-configuration, and C-18 in NSC 23925b has the R-configuration. FIG. 15 shows the three-dimensional supramolecular architecture of NSC 23925b viewed along the a-axis direction (as determined by x-ray crystallography).

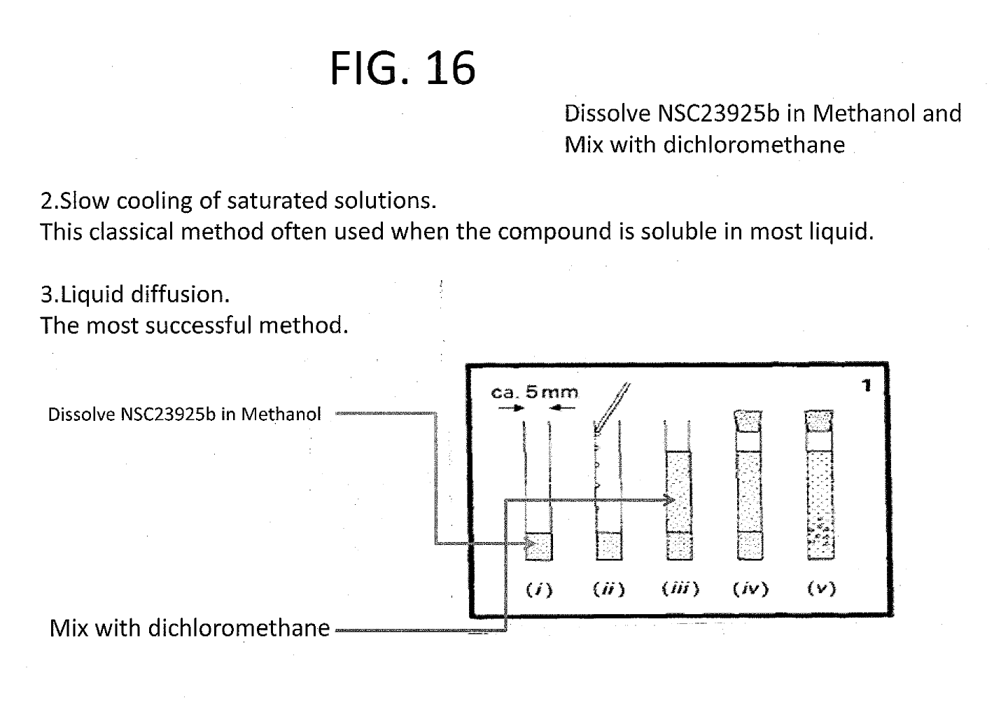

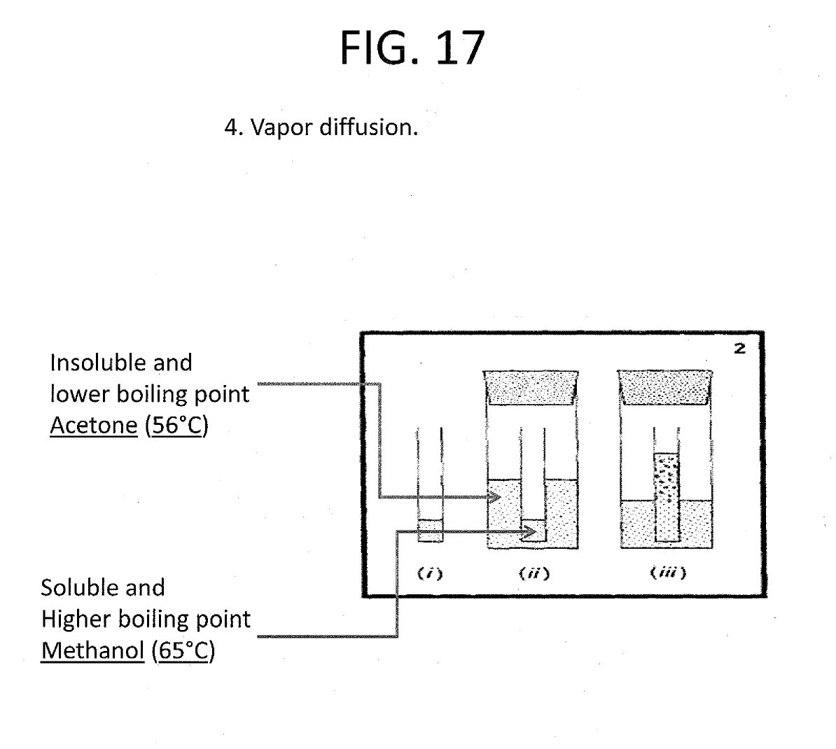

FIGS. 16 and 17 are flow diagrams that summarize methods used to obtain NSC 23925b in crystalline form for use in the x-ray crystallography.

In addition, solvent evaporation techniques were explored: place a small amount of sample in a vial, dissolve in a minimum amount of solvent, cover the opening with lab-film, pierce once with a narrow needle, leave the sample in a place that is free from vibrations and not in direct sunlight or next to a source of heat. Allow solvent to evaporate slowly; do not allow to go to dryness. NSC 23925b was found to be soluble in methanol, but not soluble in either, ethyl acetate, chloroform, dichloromethane, acetone, tetrahydrofuran, cyclohexane, cyclopentane, hexanes, and pentane.

Table 6 provides a summary of physical properties of NSC23925a, NSC23925b, NSC23925c, and NSC23925d.

TABLE 6

| | NSC23925a | NSC23925b | NSC23925c | NSC23925d |
|---|---|---|---|---|
| 5 milligram | 650 | 450 | 150 | 150 |
| solubility in | microliters | microliters | microliters | microliters |
| methanol | | | | |
| color | yellow | yellow | yellow | yellow |
| Shape of | Cubic-like | Cubic-like | Needle-like | Needle-like |
| crystal | | | | |

### Example 12

### Additional Biological Data Using Ovarian Carcinoma Cell Lines and Taxol

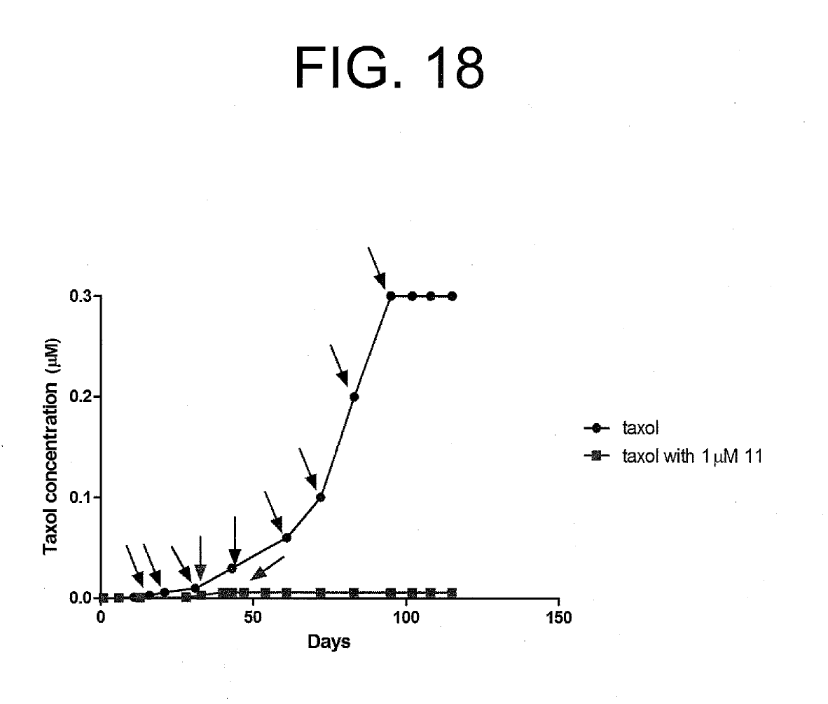

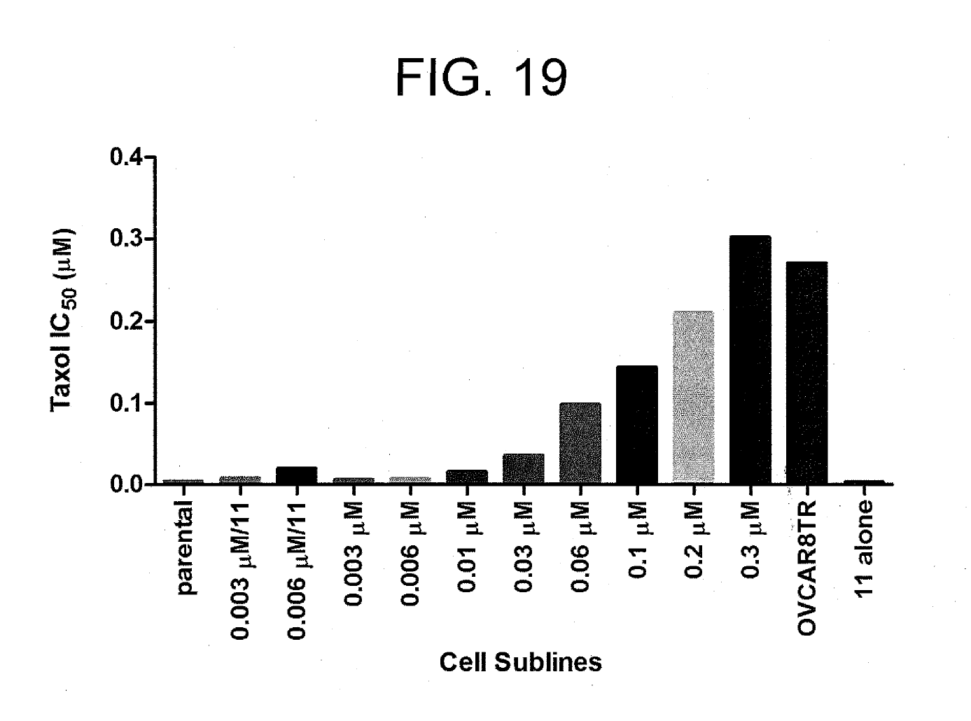

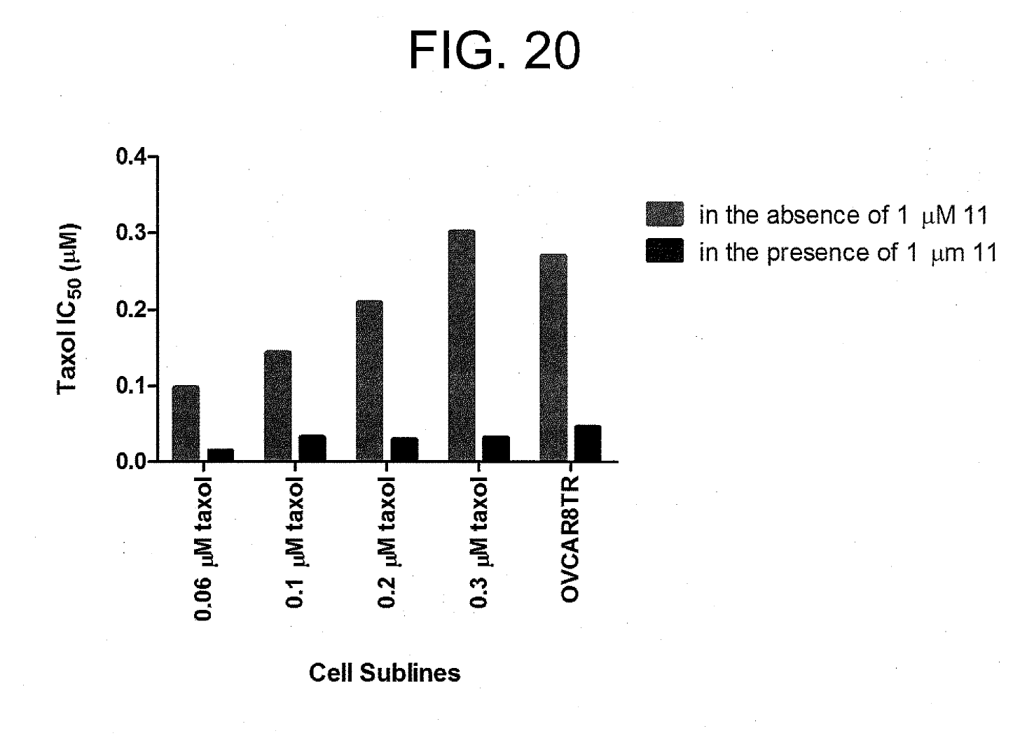

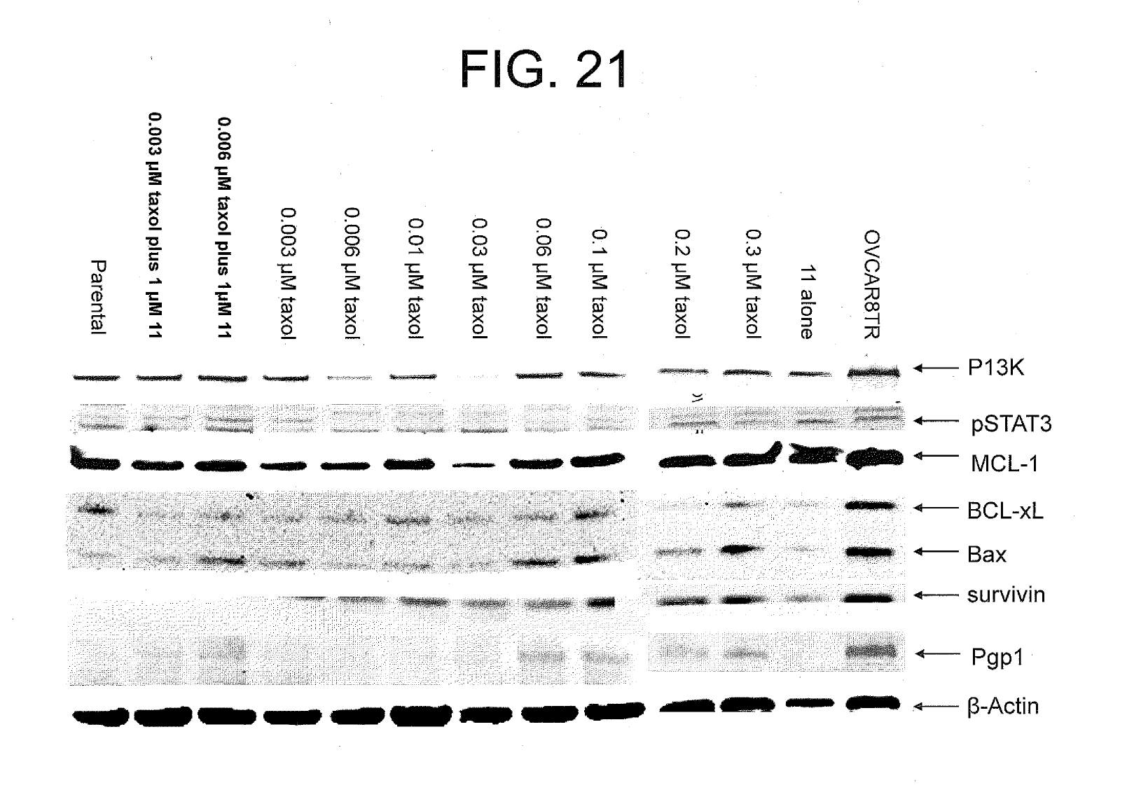

FIG. 18 is a graph showing the time course of the development of drug resistance in OVCAR8 (ovarian carcinoma) cell lines elected with taxol alone (round dots) or taxol with 1 mM compound 11 (NSC 23925b) (squares). FIG. 19 is a graph showing IC₅₀ of taxol in parental, OVCAR8TR, and different selected cells with and without compound 11 (NSC 23925b). FIG. 20 is a graph showing the effects of compound 11 (NSC 23925b) on reverse drug resistance in different selected OVCAR8 cell sublines. FIG. 21 shows, in part, related proteins level in selected cell sublines.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A substantially diastereomerically pure, optical isomer of a compound having the formula: 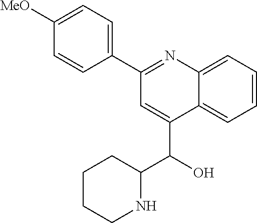 [SMILES: COc1ccc(-c2cc(C(O)C3CCCCN3)c3ccccc3n2)cc1]
and having an optical rotation of about [α]_{D}²⁰=+15.0 at a concentration of 0.2 grams/100 mL of methanol,
a compound having the formula: 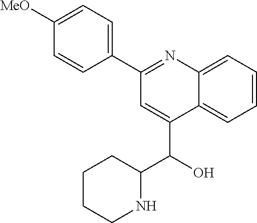 [SMILES: COc1ccc(-c2cc(C(O)C3CCCCN3)c3ccccc3n2)cc1]
and having an optical rotation of about [α]_{D}²⁰=−15.8 at a concentration of 0.2 grams/100 mL of methanol; or
a compound having the formula: 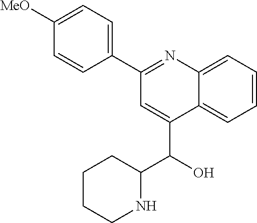 [SMILES: COc1ccc(-c2cc(C(O)C3CCCCN3)c3ccccc3n2)cc1]
and having an optical rotation of about [α]_{D}²⁰=+15.6 at a concentration of 0.2 grams/100 mL of methanol
or a pharmaceutically acceptable salt thereof, or
having the formula: 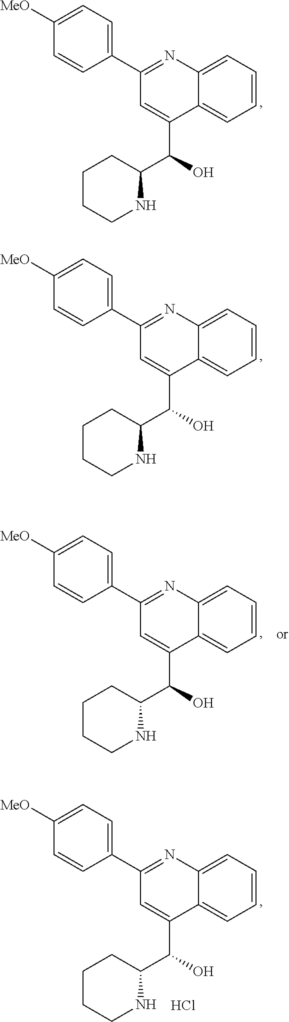 [SMILES: COc1ccc(-c2cc([C@@H](O)[C@@H]3CCCCN3)c3ccccc3n2)cc1 ; COc1ccc(-c2cc([C@H](O)[C@@H]3CCCCN3)c3ccccc3n2)cc1 ; COc1ccc(-c2cc([C@@H](O)[C@H]3CCCCN3)c3ccccc3n2)cc1 ; COc1ccc(-c2cc([C@H](O)[C@H]3CCCCN3)c3ccccc3n2)cc1 ; Cl]
or a pharmaceutically acceptable salt thereof, which is substantially free of its enantiomer and its two corresponding diastereomers.

2.-3. (canceled)

4. The compound of claim 1, or a pharmaceutically acceptable salt thereof, having an optical purity of at least 90% diastereomeric excess.

5. The compound of claim 1, or a pharmaceutically acceptable salt thereof, having an optical purity of at least 95% diastereomeric excess.

6. A pharmaceutical composition comprising a compound of claim 1, or a pharmaceutically acceptable salt thereof, and at least one pharmaceutically acceptable carrier.

7. The pharmaceutical composition of claim 6, wherein said composition further comprises an anti-cancer therapeutic agent.

8. The pharmaceutical composition of claim 7, wherein said anti-cancer therapeutic agent is selected from the group consisting of asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, and zalypsis.

9. The pharmaceutical composition of claim 8, wherein said anti-cancer therapeutic agent is selected from the group consisting of paclitaxel, doxorubicin, docetaxel, calcein-AM, daunorubicin, gemcitabine, rhodamine-123, ET-743, vincristin and zalypsis.

10. A method of reducing drug resistance in a subject diagnosed with cancer, the method comprising administering to the subject a therapeutically effective amount of a compound according to claim 1, or a pharmaceutically acceptable salt thereof.

11. (canceled)

12. The methods according to claim 10, wherein the subject is a patient undergoing cancer treatment, or wherein the method further comprises administering to the subject an anticancer therapeutic agent.

13. (canceled)

14. The method of claim 12, wherein the compound, or pharmaceutically acceptable salt thereof, and said anti-cancer therapeutic agent are administered simultaneously; wherein the compound, or pharmaceutically acceptable salt thereof, is administered to the subject prior to the administration of said anti-cancer therapeutic agent; or wherein the compound, or pharmaceutically acceptable salt thereof, is administered to the subject after the administration of said anti-cancer therapeutic agent.

15.-16. (canceled)

17. The method of claim 12, wherein said anti-cancer therapeutic agent is selected from the group consisting of asparaginase, bleomycin, calcein-AM, carboplatin, carmustine, chlorambucil, cisplatin, colaspase, cyclophosphamide, cytarabine, dacarbazine, dactinomycin, daunorubicin, docetaxel, doxorubicin (adriamycine), epirubicin, etoposide, ET-743, 5-fluorouracil, gemcitabine, hexamethylmelamine, hydroxyurea, ifosfamide, irinotecan, leucovorin, lomustine, mechlorethamine, 6-mercaptopurine, mesna, methotrexate, mitomycin C, mitoxantrone, paclitaxel, prednisolone, prednisone, procarbazine, raloxifen, rhodamine-123, streptozocin, tamoxifen, thioguanine, topotecan, vinblastine, vincristine, vindesine, and zalypsis.

18. The method of claim 17, wherein said anti-cancer therapeutic agent is selected from the group consisting of paclitaxel, doxorubicin, docetaxel, calcein-AM, daunorubicin, gemcitabine, rhodamine-I 23, ET-743, vincristin and zalypsis.

19. The method of claim 12, wherein said anti-cancer therapeutic agent is a cytotoxic drug selected from the group consisting of anthracyclines, vinca alkaloids and taxanes.

20. The method of claim 13, wherein said cancer treatment is for a cancer of breast, respiratory tract, brain, reproductive organs, digestive tract, urinary tract, eye, liver, skin, head and neck, thyroid, parathyroid or a distant metastasis of a solid tumor.

21. The method of claim 20, wherein said distant metastasis of a solid tumor is sarcoma.

22. A kit comprising an effective amount of a compound of claim 1, or pharmaceutically acceptable salt thereof, and instructions for administering said compound or pharmaceutically acceptable salt thereof for reducing drug resistance in a subject undergoing a cancer treatment, and optionally further comprising an anti-cancer therapeutic agent.

23. (canceled)

24. A method of treating cancer in a subject, said method comprising: identifying a subject having developed or susceptible to developing drug resistance; administering to said subject an effective amount of a compound of claim 1, or a pharmaceutically acceptable salt thereof, wherein said effective amount is sufficient to reduce drug resistance in the subject, thereby treating cancer in the subject.

25. The method of claim 24, wherein the subject is a patient undergoing cancer treatment.

26. The method of claim 25, wherein said method further comprises discontinuing said cancer treatment, prior to or after the administration of the compound, or pharmaceutically acceptable salt thereof.

27.-28. (canceled)

29. The method of claim 25, wherein said method further comprises administering to said subject a subsequent cancer treatment after the discontinuation of said cancer treatment and the administration of the compound, or pharmaceutically acceptable salt thereof, wherein said subsequent cancer treatment is the same as or different from said discontinued cancer treatment.

30.-31. (canceled)

32. The method of claim 29, further comprising administering to said subject one or more additional effective amounts of the compound, or pharmaceutically acceptable salt thereof, after administration of said subsequent cancer treatment.

33. The method of claim 24, wherein said cancer treatment is selected from the group consisting of surgery, chemotherapy, radiation therapy, immunotherapy and monoclonal antibody therapy.

34.-37. (canceled)
